(19) United States
(12) Patent Application Publication
(10) Pub. No.: US 2023/0338413 A1
(43) Pub. Date: Oct. 26, 2023

(54) ORAL DELIVERY OF HEPARINS

(71) Applicant: GUANGZHOU DAZHOU BIOMEDICINE LTD., Guangzhou (CN)

(72) Inventors: Steven DINH, Miami, FL (US); Huadong TANG, McLean, VA (US); Wenfei LIANG, Guangzhou (CN)

(21) Appl. No.: 18/075,640

(22) Filed: Dec. 6, 2022

(30) Foreign Application Priority Data
Dec. 6, 2021 (WO) PCT/CN2021/135770

(51) Int. Cl. A61K 31/727; A61K 47/18; A61K 9/00

(52) U.S. Cl. CPC A61K 31/727; A61K 47/183; A61K 9/0053; A61K 47/18

(57) ABSTRACT

Provided herein are pharmaceutical compositions comprising a heparin and functional excipients that can enhance oral bioavailability of the heparin. Also provided herein are methods of preparing the pharmaceutical composition and methods of using the same for treating various diseases or disorders such as DVT.

### BACKGROUND

### Field of the Invention

In various embodiments, the present invention generally relates to oral delivery of therapeutic agents.

### Background Art

Heparin is widely used as an anticoagulant for over 90 years (Heparin and anticoagulation, Frontiers in Bioscience, Landmark, 21, 1372-1392, Jun. 1, 2016). Heparin-based drugs include unfractionated heparin (UFH), low molecular weight heparin (LMWH), and synthetic heparins. Heparin inhibits reactions that lead to the clotting of blood and the formation of fibrin clots also known as thrombus. It inhibits several activated coagulation factors, in particular, factor Xa and factor IIa, by binding to the plasma protease inhibitor antithrombin III. These medications are indicated for treatment and prophylaxis of thrombosis, embolisms and many other diseases related to clotting.

Presently, heparin is administered parenterally, either by continuous or intermittent intravenous infusion or by deep subcutaneous injection. Heparin alone is not absorbed through the gastrointestinal tract.

### BRIEF SUMMARY

There remains a need for heparin pharmaceutical compositions that can be administered orally and provide an increased heparin response, i.e., an oral heparin administration that can traverse the gastrointestinal tract and provide increased bioavailability. Such a composition would provide a more convenient dosage method for patients receiving heparin. Oral heparin would also increase patient compliance, particularly, for patients requiring prophylactic therapy to prevent major venous thromboembolic events. The fact that injections and needles are not required is appealing to patients and can reduce infections due to the disruption of the integrity of the skin by injections. In addition, it can enable in home use, and can reduce the burdens of care givers (Thrombosis Journal 2006, 4:6).

In various embodiments, the present disclosure is based, in part, on the unexpected discovery that the combination of certain fatty acids and oral absorption enhancers can achieve a synergistic effect in enhancing overall oral absorption of heparin. In addition, the present disclosure is based, in part, on the unexpected discovery that the use of a chelating agent can further enhance the oral delivery of heparin.

The present disclosure solves the unmet need for heparin pharmaceutical compositions that can be administered orally and provide an increased heparin response. The pharmaceutical composition herein can provide a more convenient dosage method for patients receiving heparin. As the pharmaceutical composition herein can be orally administered to achieve an effective amount of plasma level of heparin, it can also increase patient compliance, particularly for patients requiring prophylactic therapy to prevent major venous thromboembolic events.

In some embodiments, the present disclosure provides:
[1]pharmaceutical composition comprising:
   (a) a heparin, preferably, low molecular weight heparin (LWMH);
   (b) an aliphatic acid of Formula I: RCOOH, wherein R represents an aliphatic group, or a pharmaceutically acceptable salt thereof; and
   (c) a compound of Formula II:

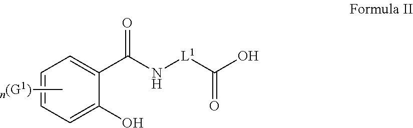

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆alkenylene.
[2] The pharmaceutical composition of [1], formulated for oral administration.
[3] The pharmaceutical composition of [1] or [2], which upon oral administration to a human subject in need thereof, delivers a therapeutically effective amount of the heparin, such as LWMH, to the human subject.
[4] The pharmaceutical composition of any of [1]-[3], wherein in Formula I, R represents an alkyl group having 1-30 carbon atoms, e.g., R is —(CH₂)₁₋₁₈CH₃,
[5] The pharmaceutical composition of any of [1]-[3], wherein in Formula I, R represents an alkyl group having 3-20 carbon atoms.
[6] The pharmaceutical composition of any of [1]-[3], wherein in Formula I, R represents an alkyl group having 5-16 carbon atoms.
[7] The pharmaceutical composition of any of [1]-[3], wherein the aliphatic acid of Formula I is a linear aliphatic acid having 2 to 20 carbon atoms, such as caprylic acid, capric acid, or lauric acid.
[8] The pharmaceutical composition of any of [1]-[7], wherein in Formula II n is 0.
[9] The pharmaceutical composition of any of [1]-[7], wherein in Formula II, n is 1 and G¹ is halogen, C₁₋₄ alkyl, or C₁₋₄ alkoxy.
[10] The pharmaceutical composition of any of [1]-[7] wherein in Formula II, n is 1 and G¹ is Cl or OCH₃.
[11] The pharmaceutical composition of any of [1]-[10], wherein in Formula I substituted or unsubstituted C₂-C₁₆alkylene.
[12] The pharmaceutical composition of any of [1]-[10] wherein in Formula II, L¹ is an unsubstituted C₃-C₁₅alkylene,
[13] The pharmaceutical composition of any of [1]-[10], wherein in Formula II, L¹ is an unsubstituted C₅-C₁₃ alkylene.
[14] The pharmaceutical composition of any of [1]-[10] wherein in Formula II, L¹ is an unsubstituted, straight-chained C₅-C₉ alkylene.
[15] The pharmaceutical composition of any of [1]-[7], wherein the compound of Formula II is

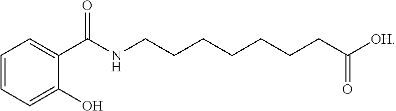 [SMILES: O=C(O)CCCCCCCNC(=O)c1ccccc1O]

[16] The pharmaceutical composition of any of [1]-[7], wherein the compound of Formula II is

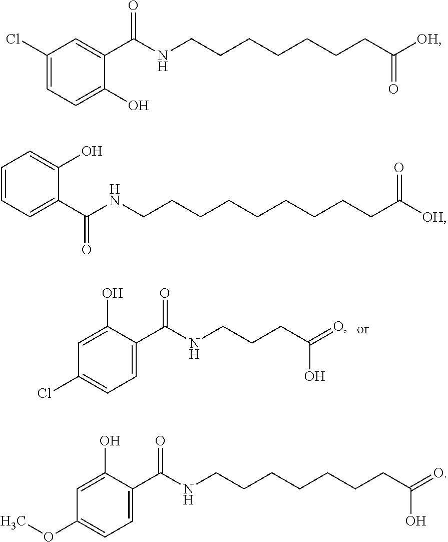 [SMILES: O=C(O)CCCCCCCNC(=O)c1cc(Cl)ccc1O ; O=C(O)CCCCCCCCCNC(=O)c1ccccc1O ; O=C(O)CCCNC(=O)c1ccc(Cl)cc1O ; COc1ccc(C(=O)NCCCCCCCC(=O)O)c(O)c1]

[17] The pharmaceutical composition of any of [1]-[16], comprising a sodium salt of the compound of Formula II.
[18] The pharmaceutical composition of any of [1]-[3], comprising a sodium salt of

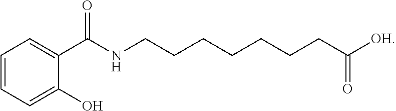 [SMILES: O=C(O)CCCCCCCNC(=O)c1ccccc1O]

[19] The pharmaceutical composition of [18], wherein the aliphatic acid of Formula I is capric acid.
[20] The pharmaceutical composition of any of [1]-[19], wherein the weight ratio of (b) the aliphatic acid of Formula I or pharmaceutically acceptable salt thereof to (c) the compound of Formula II or pharmaceutically acceptable salt thereof, (b)/(c), ranges from about 20:1 to about 1:20, such as 5:1 to 1:5, e.g., about 2:1 to about 1:2, such as about 2:1, about 1:1, or about 1:2, or any range in between.
[21] The pharmaceutical composition of any of [1]-[20], wherein the aliphatic acid of Formula I or pharmaceutically acceptable salt thereof is in an amount of about 50 mg to about 300 mg per unit dose.
[22] The pharmaceutical composition of any of [1]-[21], where the compound of Formula H or pharmaceutically acceptable salt thereof is in an amount of about 200 mg to about 400 mg per unit dose.
[23] The pharmaceutical composition of any of [1]-[22], comprising a synergistic combination of (b) the aliphatic acid of Formula I or pharmaceutically acceptable salt thereof and (c) the compound of Formula II or pharmaceutically acceptable salt thereof, for achieving enhanced oral delivery of the heparin, such as LMWH.
[24] The pharmaceutical composition of any of [1]-[23], further comprising (d): a chelating agent.
[25] The pharmaceutical composition of [24], wherein the chelating agent is an aminocarboxylic acid or a salt thereof.
[26] The pharmaceutical composition of [24], wherein the chelating agent is ethylenediaminetetraacetic acid or a salt thereof such as sodium salt.
[27] The pharmaceutical composition of any of [24]-[27], wherein the weight ratio of (b) the aliphatic acid of Formula I or pharmaceutically acceptable salt thereof to (d) the chelating agent, (b)/(d), ranges from about 20:1 to about 1:20, such as 10:1 to 1:1, e.g. about 3:1.
[28] The pharmaceutical composition of any of [24]-[27], wherein the chelating agent is in an amount of about 10 mg to about 150 mg per unit dose, preferably, about 30 mg to about 100 mg per unit dose.
[29] The pharmaceutical composition of any of [24]-[27], wherein the chelating agent is in an amount effective in further enhancing oral delivery of the heparin, such as LMWH.
[30] The pharmaceutical composition of any of [1]-[29], wherein the heparin is an LMWH, for example, the LMWH has an average molecular weight of about 3000 Daltons to about 7000 Daltons.
[31] The pharmaceutical composition of any of [1]-[29], wherein the LMWH is enoxaparin, bemipatin, nadroparin, reviparin, pamaparin, certoparin, daiteparin, or tinzaparin.
[32] The pharmaceutical composition of any of [1]-[31], further comprising a lubricant, a binder, and/or a filler.
[33] The pharmaceutical composition of any of [1]-[32], in the form of a solid oral dosage form, such as capsule or tablet, such as an entetic coated tablet, a bilayer or multilayer tablet, etc.
[34] The pharmaceutical composition of any of [24]-[33], which comprises a layer comprising a mixture of the heparin (e.g. LMWH), the aliphatic acid of Formula I or pharmaceutically acceptable salt thereof, the compound of Formula II or pharmaceutically acceptable salt thereof, and the chelating agent.
[35] The pharmaceutical composition of any of [24]-[33], which has a bilayer or multilayer structure, wherein a first layer comprises a mixture of the heparin (e.g., LMWH), the aliphatic acid of Formula I or pharmaceutically acceptable salt thereof, and the compound of Formula II or pharmaceutically acceptable salt thereof, and a second layer comprises the chelating agent, wherein the first and second layers are not the same layer.
[36] The pharmaceutical composition of any of [24]-[33], which comprises a core comprising a mixture of the heparin (e.g., LMWH), the aliphatic acid of Formula I or pharmaceutically acceptable salt thereof, and the compound of Formula II or pharmaceutically acceptable salt thereof, wherein the core is encapsulated at least partially by a shell comprising the chelating agent.
[37] The pharmaceutical composition of any of [24]-[33], which comprises particles having a core comprising a mixture of the heparin (e.g., LMWH), the aliphatic acid of Formula I or pharmaceutically acceptable salt thereof, and the compound of Formula II or pharmaceutically acceptable salt thereof, wherein the core is encapsulated at least partially by a shell comprising the chelating agent.
[38] A method of treating a disease or disorder for which administering heparin (e.g., LWMH) is beneficial, e.g., deep vein thrombosis (DVT), Sickle Cell Disease (SCD), pre-thrombotic state/recurrent spontaneous abortion (PTS/RSA), cancer associated thrombosis (CAT), dyslipidemia, etc., in a subject in need thereof, the method comprising orally administering the pharmaceutical composition of any of [1]-[37] to deliver a therapeutically effective amount of the heparin (e.g., LMWH) to the subject.
[39] A method of preparing the pharmaceutical composition of any of [1]-[37], the method comprising (a) mixing the heparin (e.g., LMWH) with the compound of Formula II or pharmaceutically acceptable salt thereof, and the aliphatic acid of Formula I or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.
[40] A method of preparing the pharmaceutical composition of any of [24]-[37], the method comprising (a) mixing the with the compound of Formula II or pharmaceutically acceptable salt thereof, and the aliphatic acid of Formula I or pharmaceutically acceptable salt thereof to form a mixture; (b) freeze-drying the mixture formed in (a) to form a freeze-dried mixture; and (c) mixing the freeze-dried mixture with the chelating agent.
[41] A method of preparing the pharmaceutical composition of any of [24]-[37], as applicable, the method comprising (a) mixing the LMWH with the compound of Formula II or pharmaceutically acceptable salt thereof, the aliphatic acid of Formula I or pharmaceutically acceptable salt thereof, and the chelating agent to form a mixture; (b) freeze-drying the mixture formed in (a) to form a freeze-dried mixture; and optionally (c) mixing the freeze-dried mixture with a pharmaceutically acceptable excipient.
[42] A method of preparing a composition comprising a heparin such as a low molecular weight heparin (LMWH), the method comprising:
   (a) mixing the heparin such as LMWH with (i) an aliphatic acid of Formula I: RCOOH, wherein R represents an aliphatic group, or a pharmaceutically acceptable salt thereof; (ii) a compound of Formula II:

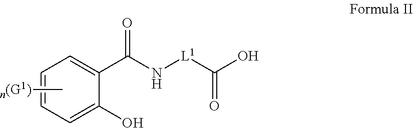

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene; and optionally (iii) a chelating agent; and
(b) freeze-drying the mixture formed in (a).
The method of [42], wherein the mixing in (a) comprises mixing the heparin (e.g., LMWH) with a sodium salt of the compound of Formula II.
[44] The method of [42] or [43]. wherein the mixing in (a) comprises mixing the heparin (e.g., LMWH) with a sodium salt of

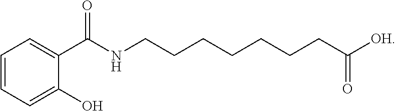 [SMILES: O=C(O)CCCCCCCNC(=O)c1ccccc1O]

[45] The method of any one of [42]-[44], wherein the aliphatic acid of Formula I is a linear aliphatic acid having 2 to 20 carbon atoms, such as caprylic acid, capric acid, or lauric acid.
[46] The method of any one of [42]-[45], wherein the mixing in (a) does not include mixing with the optional chelating agent.
[47] The method of any one of [42]-[45], wherein the mixing in (a) includes mixing with the chelating agent.
[48] The method of [47], wherein the chelating agent is ethylenediaminetetraacetic acid or a salt thereof, such as sodium salt.
[49] The method of any one of [42]-[48], wherein the heparin (e.g., LMWH) is enoxaparin, hemiparin, nadroparin, reviparin, parnaparin, certoparin, dalteparin, or tinzaparin.
[50] The method of any of [42]-[49], wherein the weight ratio of (i) the aliphatic acid of Formula I or pharmaceutically acceptable salt thereof to (ii) the compound of Formula II or pharmaceutically acceptable salt thereof, (i)/(ii), ranges from about 20:1 to about 1:20, such as 5:1 to 1:5, e.g., about 2:1 to about 1:2, such as about 2:1, about 1:1, or about 1:2, or any range in between.
[51] The method of any of [42]-[50], wherein the aliphatic acid of Formula I or pharmaceutically acceptable salt thereof is in an amount of about 50 mg to about 300 mg per unit dose.
[52] The method of any of [42]-[51], wherein the compound of Formula II or pharmaceutically acceptable salt thereof is in an amount of about 200 mg to about 400 mg per unit dose.
[53] The method of any of [42]-[52], wherein the chelating agent, if present, is in an amount of about 10 mg to about 150 mg per unit dose.
[54] The composition prepared by the method of any one of [42]-[53].
[55] A pharmaceutical composition comprising the composition of [54].
[56] A method of preparing a pharmaceutical composition comprising mixing the composition of [54] with a pharmaceutically acceptable excipient.
[57] The pharmaceutical composition prepared by the method of [55].
[58] A method of treating a disease or disorder for which administering heparin (e.g., LWMH) is beneficial, e.g., deep vein thrombosis (DVT), Sickle Cell Disease (SCD), pre-thrombotic state/recurrent spontaneous abortion (PTS/RSA), cancer associated thrombosis (CAT), etc., in a subject in need thereof, the method comprising orally administering the pharmaceutical composition of any of [55]-[57] to deliver a therapeutically effective amount of the heparin (e.g., LMWH) to the subject.
[59] A method of prophylaxis or treatment of COVID-19 infection in a subject in need thereof, the method comprising orally administering the pharmaceutical composition of any of [1]-[37] and [55]-[57] to deliver an effective amount of the heparin (e.g., LMWH) to the subject.

It is to be understood that both the foregoing summary and the following detailed description are exemplary and explanatory only, and are not restrictive of the invention herein.

### BRIEF DESCRIPTION OF THE FIGURES

FIG. 1A shows a two-layer tablet design.

FIG. 1B shows a three-layer tablet design.

FIG. 1C shows an extended two-layer tablet design.

FIG. 2 shows the anti-Xa strength vs. time profile following oral administration of Formulation 1 .

FIG. 3 shows a comparison of the anti-Xa strength vs. time profile following oral administration of Formulation 1 or Formulation 2.

FIG. 4 shows a comparison of the anti-Xa strength vs. time profile following oral administration of Formulation 1, 3A, or 3B.

FIG. 5 shows a comparison of the anti-Xa strength vs. time profile following oral administration of Formulation 1 or 4.

FIG. 6 shows a comparison of the anti-Xa strength vs. time profile following oral administration of Formulation 1 or 5.

FIG. 7 shows a comparison of the anti-Xa strength vs. time profile following oral administration of Formulation 6A, 6B, or 6C.

FIG. 8 shows a comparison of the anti-Xa strength vs. time profile following oral administration of Formulation 6A or 7.

FIG. 9 shows a comparison of the anti-Xa strength vs. time profile following oral administration of Formulation 8A, 8B, or C.

FIG. 10 shows a comparison of the anti-Xa strength vs. time profile following oral administration of Formulation 9A, 9B, or 9C.

FIG. 11 shows an anti-Xa strength vs. time profile following a subcutaneous injection of enoxaparin.

### DETAILED DESCRIPTION

The present disclosure generally relates to oral delivery of heparins. As detailed herein, the present inventors have discovered that the combination of heparins such as LMWH with SNAC and sodium caprate produced an unexpected higher drug absorption than formulations containing heparin and SNAC or sodium caprate alone. In addition, it was found that the addition of a chelating agent such as EDTA can provide additional enhancement of oral delivery of heparins.

In a broad aspect, the present disclosure provides a pharmaceutical composition comprising a heparin and one or more, particularly, two or more, functional excipients (e.g., any of those described herein). Unless otherwise contrary from context, functional excipients as used herein refer to those excipients that can enhance the oral bioavailability of heparin. For example, in some embodiments, the functional excipients refer to those that can increase the bioavailability of heparin of a composition following oral administration.

Typically, the pharmaceutical composition comprises two or more functional excipients that can synergistically enhance the oral absorption of the heparin. In some embodiments, the two or more functional excipients include an aliphatic acid of Formula I: RCOOH, wherein R represents an aliphatic group, or a pharmaceutically acceptable salt thereof. In some embodiments, the two or more functional excipients include a compound of Formula II:

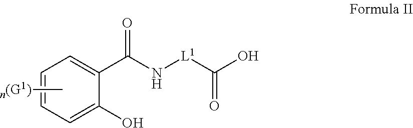

or a pharmaceutically acceptable salt thereof, wherein:
n is an integer selected from 0, 1, 2, 3, or 4;
G¹ at each occurrence is independently OH, NH₂, NH(C₁₋₄ alkyl) N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted alkylene, or substituted or unsubstituted C₂-C₁₆ alkenylene. Typically, the pharmaceutical composition is formulated for oral administration. Preferably, a therapeutically effective plasma concentration of the hepatin can be achieved following oral administration of the pharmaceutical composition herein.

In some embodiments, the present disclosure provides a pharmaceutical composition comprises (a) a hepatin, prefer-ably, a low molecular weight heparin (LWMH), (b) an aliphatic acid of Formula 1: RCOOH, wherein R represents an aliphatic group, or a pharmaceutically acceptable salt thereof, and (c) a compound of Formula II:

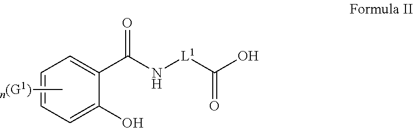

or a pharmaceutically acceptable salt thereof, wherein:
n is an integer selected from 0, 1, 2, 3, or 4;
G¹ at each occurrence is independently OH, NH₂, NH(C₁₋₄ alkyl) N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene.

### Heparins

The term “heparin” as used herein refers to all forms of heparin, including, but not limited to, unfractionated heparin, heparinoids, dermatans, chondroitins, low molecular weight heparin (e.g., enoxaparin (including enoxaparin sodium), tinzaparin (including tinzaparin sodium)), very low molecular weight heparin, and ultra low molecular weight heparin. Non-limiting examples include unfractionated heparin, such as heparin sodium (e.g., heparin sodium USP, available from Scientific Protein Labs of Waunakee, Wis.). Heparin generally has a molecular weight of from about 1,000 or 5,000 to about 30,000 Daltons. The term “low molecular weight heparin” generally refers to heparin having an average molecular weight of less than 8000 Da and for which at least 60% of all chains have a molecular weight less than 8000 Da. Non-limiting examples of low molecular weight heparin include tinzaparin, enoxaprin, and daltiparin. Tinzaparin has been approved by the U.S. Food & Drug Administration for the treatment of acute symptomatic deep vein thrombosis with or without pulmonary embolism when administered in conjunction with warfarin sodium. The sodium salt of tinazaparin is available under the trademark Innohep™ from Pharmion Corporation of Boulder, Colo. The term “very low molecular weight heparin” generally refers to heparin in which at least about 80% (by weight) of the heparin has a molecular weight of between about 1500 and about 5000 daltons. A non-limiting example of very low molecular weight heparin is bemiparin. The term “ultra low molecular weight heparin” generally refers to heparin in which at least about 80% (by weight) of the heparin has a molecular weight of between about 1000 and about 2000 daltons. A non-limiting examples of ultra low molecular weight heparin is fondiparinux.

The pharmaceutical compositions herein (e.g., any of those according to [1]-[37] and [55]-[57] as described in the Brief Summary Section, as applicable) are not limited to any particular types of heparins. Rather, the pharmaceutical compositions herein can include any of the heparin-based drugs, which include for example, unfractionated heparin (UFH), low molecular weight heparin (LMWH), and synthetic heparins.

Unfractionated heparin is a highly sulfated polysaccharide with an average molecular weight of between 15 and 19 kDa (R. J. Linhardt: Heparin: structure and activity. Journal of medicinal chemistry 46,2551-2564 (2003)). Heparin is biosynthesized in the endoplasmic reticulum and the Golgi of mast cells that are present in larger numbers in the liver, intestines, and lungs. It is extracted from food animal sources including cows and pigs, with porcine intestinal mucosa being the standard species and tissue source. Heparin binds to AT, as serine protease inhibitor, and targets coagulation proteins including factor Xa, and factor IIa (thrombin). AT binds a variably sulfated pentasaccharide sequence having a central 3-O-sulfoglucosamineresidue. AT bound to heparin undergoes a conformational change, exposing a reactive loop that is acted upon and by factor Xa and thrombin catalyzing their inactivation.

LMWH consists of smaller fragmented heparin molecules prepared through the controlled chemical or enzymatic depolymerization of unfractionated heparin (UFH) (J. Hirsh, S. S. Anand, J. L. Halperin., V, Fuster and A. H. Association: Guide to anticoagulant therapy: Heparin: a statement for healthcare professionals from the American Heart Association. Circulation 103, 2994-3018 (2001)). The depolymerization method in the production process affects generated LMWH's properties. More than ten LMWHs have been clinically used and they display similar biological properties, such as daiteparin, sodium, enoxaparin, and tinzaparin, etc. Most LMWHs have an average molecular weight between 4-5 kDa, a longer plasma half-life, better bioavailability at low doses, as well as a more predictable dose response characteristic than UFH. This allows out patient subcutaneous treatment with instead of inpatient intravenous administration of UFH. LMWHs also show low non-specific binding to macrophages, endothelial cells, platelets, osteoblasts, platelet factor 4 (PF4), and nonspecific binding to plasma proteins reducing many of the problems associated with heparin like shorter plasma half-lives, heparin induced thrombocytopenia (HIT), and osteoporosis.

ULMWHs, such as fondaparinux, are even smaller heparin chains, many being homogenous compounds, ranging in size from 1.5.-3.5. kDa (Z, Liu, S. Ji, Sheng and F., Wang: Pharmacological effects and clinical applications of ultra low molecular weight heparins. Drug Discoveries & Therapeutics 8, 1-10 (2014)). Fondaparinux is a synthetic pentasaccharide factor Xa inhibitor, Fondaparinux binds antithrombin and facilitates its inhibition of factor Xa. The advantages of ULMWHs include a higher degree of bioavailability, longer plasma half-lives, lower bleeding risk, lower risk of osteoporosis, and penetration of the blood brain barrier. ULMWHs are pure Factro Xa inhibitors, having high anti-Xa activity but no anti-IIa activity. Although these ULMWHs have some significant benefits such as no substantial binding to PF4, their drawbacks include high cost and inability to be removed by other means than renal clearance.

In some preferred embodiments, the heparin of the pharmaceutical compositions herein (e.g., any of those according to [1]-[37] and [55]-[57] as described in the Brief Summary Section, as applicable) can be a low molecular weight heparin (LMWH). In some specific embodiments, the LMWH has an average molecular weight of about 3000 Daltons to about 7000 Daltons. In some embodiments, the LMWH can be enoxaparin, bemiparin, nadroparin, reviparin, parnaparin, certoparin, dalteparin, or tinzaparin.

For example, in some preferred embodiments, the heparin of the pharmaceutical compositions herein can be enoxaparin. Enoxaparin is presently marketed in the United States, for example, under the brandname Lovenox, in the form of enoxaparin sodium in a sterile aqueous solution. Enoxaparin sodium can be obtained by alkaline depolymerization of heparin benzyl ester derived from porcine intestinal mucosa. The structure of enoxaparin sodium is characterized by a 2-O-sulfo-4-enepyranosuronic acid group at the non-reducing end and a 2-N,6-O-disulfo-D-glucosamine at the reducing end of the chain. About 20% (ranging between 15% and 25%) of the enoxaparin structure contains a 1,6 anhydro derivative on the reducing end of the polysaccharide chain.

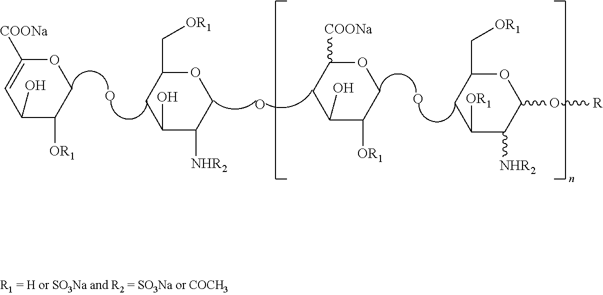

| R | X* = 15 to 25% | 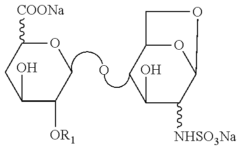 | n = 0 to 20 |
|---|---|---|---|
| | 100-X | H | n = 1 to |
| | | | 21 |

*X = Percent of polysaccharide chain containing 1,6 anhydro derivative on the reducing end

The drug substance of enoxaparin in Lovenox is the sodium salt. The average molecular weight is about 4500 daltons, According to the United States Food and Drug Administration's (“FDA”) approved label for Lovenox, the molecular weight distribution of enoxaparin is:
<2000 daltons≤20%
2000 to 8000 daltons≥68%
>8000 daltons≤18%

In some embodiments, the heparin in the pharmaceutical compositions herein (e.g., any of those according to [1]-[37] and [55]-[57] as described in the Brief Summary Section, as applicable) can be enoxaparin sodium.

In some embodiments, the heparin in the pharmaceutical compositions herein can be dalteparin, such as dalteparin sodium. Dalteparin sodium can be produced through controlled nitrous acid depolymerization of sodium heparin from porcine intestinal mucosa followed by a chromatographic purification process. It is composed of strongly acidic sulfated polysaccharide chains (oligosaccharide, containing 2,5-anhydro-D-mannitol residues as end groups) with an average molecular weight of 5,000 and about 90% of the material within the range 2,000-9,000. According to the FDA approved label for Fragmin (dalteparin sodium injection), the molecular weight distribution of enoxaparin is:
<3000 daltons 3.0-15%
3,000 to 8,000 daltons 65.0-78,0%; and
>8,000 daltons 14.0-26.0%

The structure of dalteparin sodium can be represented by the chemical formula shown below according to the FDA approved label for Fragmin:

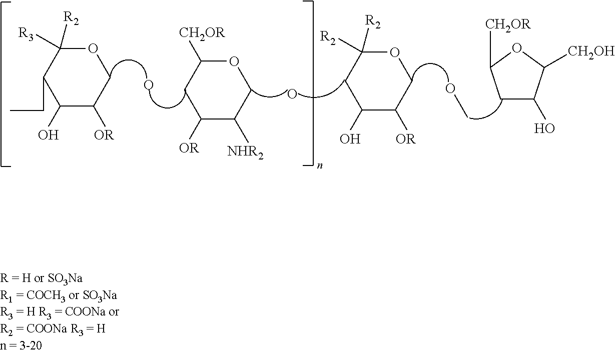

In some embodiments, the heparin in the pharmaceutical compositions herein can be tinzaparin, such as tinzaparin sodium. Tinzaparin sodium is the sodium salt of a low molecular weight heparin that can be obtained by controlled enzymatic depolymerization of heparin from porcine intestinal mucosa using heparinase from Flavobacterium heparinum. The majority of the components have a 2-O-sulpho-4-enepyranosuronic acid structure at the non-reducing end and a 2-N,6-O-disulpho-D-glucosamine structure at the reducing end of the chain. The average molecular weight ranges between 5,500 and 7.500 daltons. The molecular weight distribution is:
<2,000 Daltons<10%
2,000 to 8,000 Daltons 60% to 72%
>8,000 Daltons 22% to 36%

The structure of tinzaparin sodium can be represented by the chemical formula shown below according to the FDA approved label for Innohep (tinzaparin sodium injection).

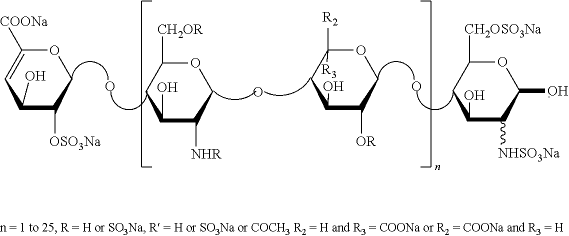

### Aliphatic Acid of Formula I

Typically, the pharmaceutical composition herein comprises an aliphatic acid of Formula I: RCOOH, wherein R represents an aliphatic group, or a pharmaceutically acceptable salt thereof.

Useful aliphatic acids are not particularly limited. For example, in some embodiments, the aliphatic acid has a Formula I: RCOOH, wherein R represents an alkyl group having 1-30 carbon atoms. The alkyl group can be a linear or branched chain alkyl group. For example, in some embodiments, R in Formula I can be —(CH₂)₁₋₁₈CH₃. In some embodiments, R in Formula I can be an alkyl group having 3-20 carbon atoms. In some embodiments, R in Formula I can be an alkyl group having 5-16 carbon atoms. In some embodiments, the aliphatic acid of Formula I is a linear aliphatic acid having 2 to 20 carbon atoms, such as caprylic acid, capric acid, or lauric acid. In any of the embodiments described herein, unless otherwise specified or contrary from context, the aliphatic acid of Formula I can be capric acid.

The aliphatic acid of Formula I can be present in the pharmaceutical composition herein as a free acid or any pharmaceutically acceptable salt thereof, such as an alkali or alkaline salt thereof, for example, a sodium or potassium salt. In some preferred embodiments, the pharmaceutical composition herein comprises sodium caprate.

### Compound of Formula 11

Typically, the pharmaceutical composition herein comprises a compound of Formula II:

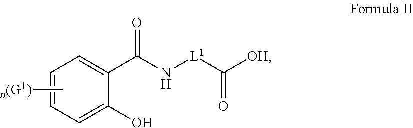

or a pharmaceutically acceptable salt thereof, wherein the variables are defined herein.

In some embodiments, the compound of Formula II can have no G¹ substituents on the phenyl ring, i.e., n is 0.

In some embodiments, the compound of Formula II can have one G¹ substituted on the phenyl ring, i.e., n is 1. In some embodiments, in Formula II, n is 1, and G¹ is a halogen, C₁₋₄ alkyl, or C₁₋₄ alkoxy. In some embodiments, in Formula II, n is 1, and G¹ is Cl. In some embodiments, in Formula II, n is 1, and G¹ is OCH₃.

L¹ in Formula II is typically a substituted or unsubstituted C₁₋₄ alkylene. For example, in some embodiments, L¹ is an unsubstituted C₃-C₁₅ alkylene. In some embodiments, L¹ is an unsubstituted C₅-C₁₃ alkylene. The alkyelene can be a straight-chained or a branched alkyelene. For example, in some embodiments, L¹ is an unsubstituted, straight-chained C₅-C₉ alkylene.

In some preferred embodiments, the compound of Formula II can be

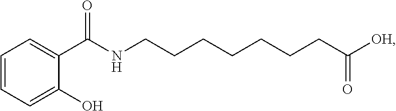 [SMILES: O=C(O)CCCCCCCNC(=O)c1ccccc1O]

which has a chemical name of 8-(2-hydroxybenzamido)octanoic acid (ChemDraw Software, version. 20.0). In preferred embodiments, the pharmaceutical composition herein comprises a salt (preferably sodium salt) of 8-(2-hydroxybenzarnido)octanoic acid, which can be prepared using the method described in e.g. WO96/030036, WO00/046182, NV001/092206 or WO2008/028859. The salt of 8-(2-hydroxybenzamido)octanoic acid (alternatively known as N-(8-(2-hydroxybenzoyl)amino)caprylic acid) may be crystalline and/or amorphous. In some embodiments the delivery agent comprises the anhydrate, monohydrate, dihydrate, trihydrate, a solvate or one third of a hydrate of the salt of N-(8-(2-hydroxybenzoyl)amino) caprylic acid as well as combinations thereof. In some embodiments, the pharmaceutical composition herein comprises a salt of N-(8-(2-hydroxybenzoyl)amino)caprylic acid as described in WO2007/121318.

In more preferred embodiments, the pharmaceutical composition comprises sodium N-(8-(2-hydroxybenzoyl)amino)caprylate (referred to as “SNAC” herein).

In some preferred embodiments, the compound of Formula II can be

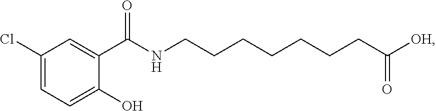 [SMILES: O=C(O)CCCCCCCNC(=O)c1cc(Cl)ccc1O]

which has a chemical name of 8-(5-chloro-2-hydroxybenzamido)octanoic acid (Chen-Draw Software, version 20.0). In some embodiments, the pharmaceutical composition herein comprises a salt of 8-(5-chloro-2-hydroxybenzamido)octanoic acid.

In some embodiments, the compound of Formula II can be

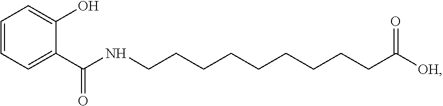 [SMILES: O=C(O)CCCCCCCCCNC(=O)c1ccccc1O]

which has a chemical name 10-((2-hydroxybenzoyl)amino)decanoic acid. In some embodiments, the pharmaceutical composition herein comprises a salt of 10-((2-hydroxybenzoyl)amino)decanoic acid, such as sodium 10-((2-hydroxybenzoyl)amino)decanoate.

In some embodiments, the compound of Formula II can be

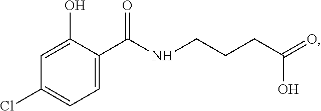 [SMILES: O=C(O)CCCNC(=O)c1ccc(Cl)cc1O]

which has a chemical name N-(4-chlorosalicyloyl)-4-aminobutyric acid. In some embodiments, the pharmaceutical composition herein comprises a salt of N-(4-chlorosalicyloyl)-4-aminobutyric acid, such as sodium N-(4-chlorosalicyloyl)-4-aminobutyrate.

In some embodiments, the. compound of Formula II can be

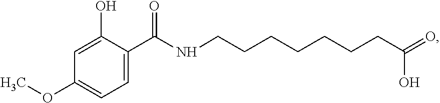 [SMILES: COc1ccc(C(=O)NCCCCCCCC(=O)O)c(O)c1]

which has a chemical name N-[8-(2-hydroxy-4-methoxy)benzoyl]amino caprylic acid. In some embodiments, the pharmaceutical composition herein comprises a salt of N-[8-(2-hydroxy-4-methoxy)benzoyl]amino caprylic acid, such as sodium N-[8-(2-hydroxy-4-methoxy)benzoyl]amino caprylate.

The combinations of heparin, aliphatic acid of Formula and the compound of Formula it are not particularly limited.

In some preferred embodiments, the pharmaceutical composition herein comprises (a) a heparin (e.g., any of those described herein, such as an LMWH, e.g., enoxaprin); (b) capric acid or a pharmaceutically acceptable salt thereof; and (c) the compound of Formula II or a pharmaceutically acceptable salt thereof.

In some preferred embodiments, the pharmaceutical composition herein comprises (a) a heparin (e.g., any of those described herein, such as an LMWH, e.g., enoxaprin); (b) a linear aliphatic acid having 2 to 20 carbon atoms, such as caprylic acid, capric acid, or lauric acid or a pharmaceutically acceptable salt thereof; and (c) SNAC.

In some preferred embodiments, the pharmaceutical composition herein comprises (a) a heparin (e.g., any of those described herein, such as an LMWH, e.g., enoxaprin); (b) capric acid or a pharmaceutically acceptable salt thereof; and (c) 8-(2-hydroxybenzamido)octanoic acid or a pharmaceutically acceptable salt thereof.

In some preferred embodiments, the pharmaceutical composition herein comprises (a) a heparin (e.g., any of those described herein, such as an LMWH, e.g., enoxaprin); (b) sodium caprate; and (c) SNAC.

In some preferred embodiments, the pharmaceutical composition herein comprises (a) enoxaparin; (b) sodium caprate; and (c) SNAC.

Typically, in the pharmaceutical compositions herein, as applicable, the weight ratio of (b) the aliphatic acid of Formula I or pharmaceutically acceptable salt thereof to (c) the compound of Formula II or pharmaceutically acceptable salt thereof, (b)/(c), ranges from about 20:1 to about 1:20, such as 5:1 to 1:5, e.g., about 2:1 to about 1:2., such as about 2:1, about 1:1, or about 1:2, or any range in between.

In some embodiments, the pharmaceutical composition comprises the aliphatic acid of Formula I or pharmaceutically acceptable salt thereof in an amount of about 50 mg to about 300 mg per unit dose, such as about 50 mg, about 100 mg, about 150 mg, about 200 mg, about 300 mg, or any range between the recited value, per unit dose. As used herein, unless otherwise specified or obviously contrary from context, the weight of the aliphatic acid of Formula 1 or pharmaceutically acceptable salt thereof should be understood as the equivalent weight expressed as the weight of the free acid. However when referring to the amount of sodium caprate specifically, the amount should be understood as the weight of the sodium salt itself, not the corresponding equivalent weight of capric acid. In some embodiments, the pharmaceutical composition comprises the aliphatic acid of Formula 1 or pharmaceutically acceptable salt thereof in an amount of at least 0.6 mmol (millimole), such as selected from the group consisting of at least 0.65 mmol, at least 0.7 mmol, at least 0.75 mmol, at least 0.8 mmol, at least 0,8 mmol, at least 0.9 mmol, at least 0.95 mmol and at least 1 mmol, per unit dose. In some embodiments, the pharmaceutical composition comprises the aliphatic acid of Formula I or pharmaceutically acceptable salt thereof in an amount of 0.6 mmol to 2 mmol, such as 0,8 mmol to 1.3 mmol, 0.9 mmol to 1.1 mmol, such as 0.95 mmol, 1.0 mmol, etc., per unit dose.

In some embodiments, the pharmaceutical composition comprises the compound of Formula II or pharmaceutically acceptable salt thereof in an amount of about 200 mg to about 400 mg per unit dose, such as about 200 mg, about 250 mg, about 300 mg, about 350 mg, about 400 mg, or any range between the recited values, per unit dose. As used herein, unless otherwise specified or obviously contrary from context, the weight of the compound of Formula II or pharmaceutically acceptable salt thereof should be understood as equivalent weight expressed as the weight of the compound of Formula II. However, when referring to the amount of SNAC specifically, the amount should be understood as the weight of the sodium salt itself, not the corresponding equivalent weight of the acid. In some embodiments, the pharmaceutical composition comprises the compound of Formula II or pharmaceutically acceptable salt thereof in an amount of at least 0.6 such as selected from the group consisting of at least 0.65 mmol, at least 0.7 mmol, at least 0.75 mmol, at least 0.8 mmol, at least 0.8 mmol, at least 0.9 mmol, at least 0.95 mmol and at least 1 mmol, per unit dose. In some embodiments, the pharmaceutical composition comprises the compound of Formula II or pharmaceutically acceptable salt thereof in an amount of 0.6 mmol to 2 mmol, such as 0.8 mmol to 1.3 mmol, or 0.9 mmol to 1.1 mmol, such as 1 mmol, per unit dose.

Typically, the pharmaceutical composition comprises a synergistic combination of (b) the aliphatic acid of Formula I or pharmaceutically acceptable salt thereof and (c) the compound of Formula II or pharmaceutically acceptable salt thereof, for achieving enhanced oral delivery of the heparin, such as the LMWH, e.g., enoxaparin.

In some specific embodiments, the pharmaceutical composition comprises, per unit dose, (a) a heparin (e.g., any of those described herein, such as an LMWH, e.g., enoxaprin); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values). The amount of the heparin is not particularly limited, for example, typically, the heparin can be in an amount of about 1 mg to about 200 mg (e.g., about 10 mg, about 50 mg, about 100 mg, about 200 mg, or any range between the recited values), per unit dose. In some embodiments, the heparin is an LMWH, which is in an amount of about 1000 anti-Xa international units (IU) to about 25,000 IU (e.g., about 2500, 5000, 7500, 10000, 12500, 15000, 18000, or any range between the recited values), per unit dose. As would be understood by a person of ordinary skill in the art, the IU herein refers to the W.H.O. First International Low Molecular Weight Heparin Reference Standard.

In some specific embodiments, the pharmaceutical composition comprises, per unit dose, (a) a heparin (e.g., any of those described herein, such as an LMWH, e.g., enoxaprin); (b) sodium caprate in an amount of about 0.6 mmol to 2. mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol). The amount of the heparin is not particularly limited, for example, typically, the heparin can be in an amount of about 1 mg to about 200 mg (e.g., about 10 mg, about 50 mg, about 100 mg, about 200 mg, or any range between the recited values), per unit dose. In some embodiments, the heparin is an LMWH, which is in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition comprises, per unit dose, (a) a low molecular weight heparin (e.g., any of those described herein); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values). The amount of the heparin is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition comprises, per unit dose, (a) a low molecular weight heparin (e.g., any of those described herein); (b) sodium caprate in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol). The amount of the heparin is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition comprises, per unit dose, (a) enoxaparin (e.g., enoxaparin sodium); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values). The amount of the enoxaparin is not particularly limited, for example, typically, the enoxaparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition comprises, per unit dose, (a) enoxaparin (e.g., enoxaparin sodium); (b) sodium caprate in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol). The amount of the enoxaparin is not particularly limited, for example, typically, the enoxaparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7:500, 10000, 12500, 15000, 18000, or any range between the recited values), per unit dose.

### Chelating Agent

In some embodiments, the pharmaceutical compositions herein can further comprise (d) a chelating agent. As shown herein, adding a chelating agent such as EDTA can further enhance the oral delivery of heparin.

The chelating agent is typically an aminocarboxylic acid or a salt thereof. In some preferred embodiments, the chelating agent is ethylenediaminetetraacetic acid (EDTA) or a salt thereof, such as sodium salt.

The amount of chelating agent is not particularly limited and can typically range from about 10 mg to about 150 mg (e.g., about 20 mg, about 30 mg, about 50 mg, about 75 mg, about 100 mg, about 150 mg, or any range between the recited values) per unit dose. Preferably, the chelating agent is included in an amount that can further enhance the oral delivery of heparin of a composition compared to substantially the same composition without the chelating agent.

For example, in any of the embodiments described herein, unless specified or otherwise obviously contrary from context, the pharmaceutical compositions herein can further comprise (d) a chelating agent which is EDTA or a salt thereof, preferably, sodium EDTA, which is present in an amount of about 30 mg to about 100 mg per unit dose, such as about 50 mg per unit dose. To be clear, when the salt form is not specified, the amount of the chelating agent should be understood as the equivalent amount of the corresponding acid form. However, the amount of “sodium EDTA” should be understood as referring to the amount based on the sodium salt itself.

In any of the embodiments described herein, unless otherwise specified or contrary from context, the pharmaceutical composition can be in the form of a solid oral dosage form. For example, the pharmaceutical composition herein can typically be a capsule or tablet. In some embodiments, the pharmaceutical composition herein can be formulated as a liquid capsule, soft-gel capsule, or another oral dosage form. In any of the embodiments described herein, unless otherwise specified or contrary from context, the pharmaceutical composition (e.g., any of those according to [1]-[37] and [55]-[57] as described in the Brief Summary Section, as applicable) can be in a unit dosage form.

The pharmaceutical composition herein can optionally include one or more further excipients, such as those suitable for oral administration. For example, in some embodiments, the pharmaceutical composition herein includes at least one pharmaceutically acceptable excipient. The term “excipient” as used herein broadly refers to any component other than the active therapeutic ingredient(s). The excipient may be an inert substance, an inactive substance, and/or a not medicinally active substance. The excipient may serve various purposes, e.g. as a carrier, vehicle, filler, binder, lubricant, glidant, disintegrant, flow control agents, crystallization retarders, solubilizers, stabilizer, colouring agent, flavouring agent, surfactant, enzyme inhibitors, basifiers, acidifiers, emulsifier and/or to improve administration, and/or absorption of the active substance, tablet coating agents to control the dissolution rates of the solid dosage form according to the pH in the GI tract. A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005). In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization retarders such as Povidone, etc.; solubilizers such as Pluronic, Povidone, etc.; colouring agents, including dyes and pigments such as Iron Oxide Red or Yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

In some embodiments, the pharmaceutical composition herein can comprise a lubricant, a binder, and/or a filler. However, in some embodiments, the pharmaceutical composition herein can also be free or substantially free of a lubricant, such as having less than 0.1% by weight, less than 0.05% by weight, less than 0.01% by weight, or non-detectable amount, of a lubricant. In some embodiments, the pharmaceutical composition herein can also be free or substantially free of a binder, such as having less than 0.1% by weight, less than 0.05% by weight, less than 0.01% by weight, or non-detectable amount, of a binder. In some embodiments, the pharmaceutical composition herein can also be free or substantially free of a filler, such as having less than 0.1% by weight, less than 0.05% by weight, less than 0.01% by weight, or non-detectable amount, of a filler.

In some embodiments, the pharmaceutical composition (e.g., any of those according to [1]-[37] and [55]-[57] as described in the Brief Summary Section, as applicable) is in the form of a unit dosage form.

### Dosage Form Structure

In some embodiments, the pharmaceutical compositions herein (e.g., any of those according to [1]-[37] and [55]-[57] as described in the Brief Summary Section, as applicable) can be characterized by having certain dosage form structures, such as tablet structures. For example, in some embodiments, the pharmaceutical composition can be a coated tablet, such as an enteric coated tablet. In some embodiments, the pharmaceutical composition can be a monolayer tablet. In some embodiments, the pharmaceutical composition can be a bilayer tablet. In some embodiments, the pharmaceutical composition can be a multilayer tablet. In some embodiments, the pharmaceutical composition can be a capsule comprising granular particles, which comprise a mixture of the heparin such as LMWH (e.g., any of those described herein), the aliphatic acid of Formula I or pharmaceutically acceptable salt thereof (e.g., any of those described herein), the compound of Formula II or pharmaceutically acceptable salt thereof (e.g., any of those described herein), and optionally the chelating agent (e.g., any of those described herein).

For example, in some embodiments, the pharmaceutical composition (e.g., any of those according to [1]-[37] and [55]-[57] as described in the Brief Summary Section, as applicable) can comprise a layer comprising a mixture of the LMWH (e.g., any of those described herein), the aliphatic acid of Formula I or pharmaceutically acceptable salt thereof (e.g., any of those described herein), the compound of Formula II or pharmaceutically acceptable salt thereof (e.g., any of those described herein), and the chelating agent (e.g., any of those described herein).

In some embodiments, the pharmaceutical composition (e.g., any of those according to [1]-[37] and [55]-[57] as described in the Brief Summary Section, as applicable) can have a bilayer or multilayer structure, wherein a first layer comprises a mixture of the LMWH (e.g., any of those described herein), the aliphatic acid of Formula I or pharmaceutically acceptable salt thereof (e.g., any of those described herein), and the compound of Formula II or pharmaceutically acceptable salt thereof (e.g., any of those described herein), and a second layer comprises the chelating agent (e.g., any of those described herein), wherein the first and second layers are not the same layer.

In some embodiments, the pharmaceutical composition can have a core comprising a mixture of the LMWH (e.g., any of those described herein), the aliphatic acid of Formula I or pharmaceutically acceptable salt thereof (e.g., any of those described herein), and the compound of Formula II or pharmaceutically acceptable salt thereof (e.g., any of those described herein), wherein the core is encapsulated at least partially by a shell comprising the chelating agent.

In some embodiments, the pharmaceutical composition can comprise particles having a core comprising a mixture of the LMWH (e.g., any of those described herein), the aliphatic acid of Formula I or pharmaceutically acceptable salt thereof (e.g., any of those described herein), and the compound of Formula II or pharmaceutically acceptable salt thereof (e.g., any of those described herein), wherein the core is encapsulated at least partially by a shell comprising the chelating agent.

For example, in some more specific embodiments, the pharmaceutical composition can be a monolayer tablet, which comprises, per unit dose, (a) a low molecular weight heparin (e.g., any of those described herein, such as enoxaparin); (b) sodium caprate in an amount of about 50 mg to about 300 mg (e.g., about 100 mg, about 150 mg, about 200 mg, or any range between the recited values); (c) SNAC in an amount of about 200 mg to about 400 mg (e.g., about 200 mg, about 300 mg, about 400 mg, or any range between the recited values); and (d) sodium EDTA in an amount of about 30 mg to about 100 mg (e.g., about 30 mg, about 50 mg, about 75 mg, about 100 mg, or any range between the recited values). The amount of the LMWH is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition can be a monolayer tablet, which comprises, per unit dose, (a) a low molecular weight heparin (e.g., any of those described herein such as enoxaparin); (b) sodium caprate in an amount of about 0.6 mmol to 2 mmol 0,6 mmol, 0.8 mmol, 0,9 mmol, 1 mmol, 1.1 mmol, 1.3 mmol, 2 mmol, or any range between the recited values, such as 0.9-1.1 mmol); (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-13 mmol); and (d) sodium EDTA in an amount of about 30 mg to about 100 mg (e.g., about 30 mg, about 50 mg, about 75 mg, about 100 mg, or any range between the recited values). The amount of the LMWH is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000 7500, 10000, 12500, 15000, 18000, or any range between the recited values), per unit dose.

In some specific embodiments, the pharmaceutical composition can be a bilayer tablet comprising a first layer and a second layer, wherein, per unit dose, the first layer comprises (a) a low molecular weight heparin (e.g., any of those described herein such as enoxaparin); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values); and the second layer comprises (d) sodium EDTA in an amount of about 30 mg to about 100 mg (e.g., about 30 mg, about 50 mg, about 75 mg, about 100 mg, or any range between the recited values), wherein the first layer and the second layer are not the same layer. In some embodiments, the first layer is free or substantially free of a chelating agent. In some embodiments, the first layer is free or substantially free of sodium EDTA. The amount of the LMWH is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition can be a bilayer tablet comprising a first layer and a second layer, wherein, per unit dose, the first layer comprises (a) a low molecular weight heparin (e.g., any of those described herein such as enoxaparin); (b) sodium caprate in an amount of about 0,6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol), and (c) SNAC in an amount of 0.6 mmol to 2 mmol (e.g., 0.6 mmol, 0 8 mmol, 0.9 mmol, 1 mmol, 1.1 mmol, 1.3 mmol, 2 mmol, or any range between the recited values, such as 0.9-1.3 mmol); and the second layer comprises (d) sodium EDTA in an amount of about 30 mg to about 100 mg (e.g., about 30 mg, about 50 mg, about 75 mg, about 100 mg, or any range between the recited values), wherein the first layer and the second layer are not the same layer. In some embodiments, the first layer is free or substantially free of a chelating agent. In some embodiments, the first layer is free or substantially free of sodium EDTA. The amount of the LMWH is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition can be a tablet comprising a first layer and one or more additional layers, wherein, per unit dose, the first layer comprises (a) a low molecular weight heparin (e.g., any of those described herein such as enoxaparin); (b) sodium caprate in an amount of about 50 mg to about 300 mg (e.g., about 100 mg, about 150 mg, about 200 mg, or any range between the recited values); and (c) SNAC in an amount of about 200 mg to about 400 mg (e.g., about 200 mg, about 300 mg, about 400 mg, or any range between the recited values); and the one or more additional layers comprise (d) sodium EDTA in an amount of about 30 mg to about 100 mg (e.g., about 30 mg, about 50 mg, about 75 mg, about 100 mg, or any range between the recited values), wherein the first layer and the one or more additional layers are not the same layer. In some embodiments, the first layer is free or substantially free of a chelating agent. In some embodiments, the first layer is free or substantially free of sodium EDTA. In some embodiment, the one or more additional layers encapsulate partially or entirely the first layer. The amount of the LMWH is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

In some specific embodiments, the pharmaceutical composition can be a tablet comprising a first layer and one or more additional layers, wherein, per unit dose, the first layer comprises (a) a low molecular weight heparin (e.g., any of those described herein such as enoxaparin); (b) sodium caprate in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol.); and (c) SNAC in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol); and the one or more additional layers comprise (d) sodium EDTA in an amount of about 30 mg to about 100 mg (e.g., about 30 mg, about 50 mg, about 75 mg, about 100 mg, or any range between the recited values), wherein the first layer and the one or more additional layers are not the same layer. In some embodiments, the first layer is free or substantially free of a chelating agent. In some embodiments, the first layer is free or substantially free of sodium EDTA. In some embodiment, the one or more additional layers encapsulate partially or entirely the first layer. The amount of the LMWH is not particularly limited, for example, typically, the heparin can be in an amount of about 1 mg to about 200 mg (e.g., about 10 mg, about 50 mg, about 100 mg, about 200 mg, or any range between the recited values) or in an amount of about 1000 anti-Xa international units (IU) to about 25,000 IU (e.g., about 2500, 5000, 7500, 10000, 12500, 15000, 18000, or any range between the recited values), per unit dose.

### Method of Preparation

The pharmaceutical compositions can be prepared by a person of ordinary skilled in the art in view of the present disclosure.

In some embodiments, the present disclosure also provides a method of preparing a pharmaceutical composition comprising a heparin. The method typically comprises: mixing the heparin (e.g., any of those described herein, such as an LMWH described herein, e.g., enoxaparin) with a compound of Formula II described herein or pharmaceutically acceptable salt thereof, and an aliphatic acid of Formula I described herein or pharmaceutically acceptable salt thereof, and optionally a chelating agent to form a mixture; and optionally mixing the mixture with a pharmaceutically acceptable excipient.

In some preferred embodiments, a freeze-drying step is used to prepare a freeze-dried mixture comprising heparin. For example, in some embodiments, the present disclosure also provides a method of preparing a pharmaceutical composition comprising a heparin, which comprises:
(a) mixing the heparin (e.g., any of those described herein, such as an LMWH described herein, e.g., enoxaparin) with a compound of Formula II described herein or pharmaceutically acceptable salt thereof, and an aliphatic acid of Formula 1 described herein or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.

In some embodiments, the heparin is mixed first with the compound of Formula II described herein or pharmaceutically acceptable salt thereof, followed by addition of the aliphatic acid of Formula I described herein or pharmaceutically acceptable salt thereof to form the mixture.

In some embodiments, the present disclosure also provides a method of preparing a pharmaceutical composition comprising a heparin, which comprises:
(a) mixing the heparin (e.g., any of those described herein, such as an LMWH described herein, e.g., enoxaparin) with a compound of Formula II described herein or pharmaceutically acceptable salt thereof, and an aliphatic acid of Formula I described herein or pharmaceutically acceptable salt thereof to form a mixture; (b) freeze-drying the mixture formed in (a) to form a freeze-dried mixture; and optionally (c) mixing the freeze-dried mixture with a chelating agent (e.g., any of those described herein, such as EDTA).

In some embodiments, the present disclosure also provides a method of preparing a pharmaceutical composition comprising a heparin, which comprises:
(a) mixing the heparin (e.g., any of those described herein, such as an LMWH described herein, e.g., enoxaparin) with a compound of Formula II described herein or pharmaceutically acceptable salt thereof, an aliphatic acid of Formula I described herein or pharmaceutically acceptable salt thereof, and a chelating agent (e.g., any of those described herein, such as EDTA) to form a mixture; (b) freeze-drying the mixture formed in (a) to form a freeze-dried mixture; and optionally (c) mixing the freeze-dried mixture with a pharmaceutically acceptable excipient.

Although in some preferred embodiments, freeze-drying a mixture of ingredients is preferred. In some embodiments, freeze-drying is not necessary.

In some embodiments, the present disclosure also provide a method of preparing a composition comprising a heparin (e.g., any of those described herein, such as an LMWH described herein, e.g., enoxaparin), the method comprising:
(c) mixing the heparin with (i) an aliphatic acid of Formula I: RCOOH, wherein R represents an aliphatic group, or a pharmaceutically acceptable salt thereof; and (ii) a compound of Formula II:

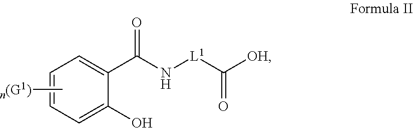

or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., C₁₋₄ alkyl, or C₁₋₄ alkoxy (e.g., OCH₃); and
L¹ is a substituted or unsubstituted C₂-C₁₆alkylene, or substituted or unsubstituted C₂-C₁₆alkenylene; and optionally (iii) a chelating agent; and
(d) freeze-drying the mixture formed in (a).

In some embodiments, the mixing in (a) comprises mixing the heparin (e.g., LMWH such as enoxaparin) with a sodium salt of the compound of Formula II. In some embodiments, the mixing in (a) comprises mixing the heparin (e.g., LMWH such as enoxaparin) with a sodium salt of

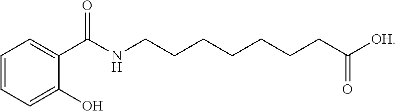 [SMILES: O=C(O)CCCCCCCNC(=O)c1ccccc1O]

In some embodiments, the aliphatic acid of Formula I is a linear aliphatic acid having 2 to 20 carbon atoms, such as caprylic acid, capric acid, or lauric acid. In some embodiments, the weight ratio of (i) the aliphatic acid of Formula I or pharmaceutically acceptable salt thereof to (ii) the compound of Formula II or pharmaceutically acceptable salt thereof, (i)/(ii), ranges from about 20:1 to about 1:20, such as 5:1 to 1:5, e.g., about 2:1 to about 1:2, such as about 2:1, about 1:1, or about 1:2, or any range in between. In some embodiments, the aliphatic acid of Formula I or pharmaceutically acceptable salt thereof is in an amount of about 50 mg to about 300 mg per unit dose. In some embodiments, the compound of Formula II or pharmaceutically acceptable salt thereof is in an amount of about 200 mg to about 400 mg per unit dose.

In some embodiments, the heparin is an LMWH.

In some embodiments, the heparin is enoxaparin, bemiparin, nadroparin, reviparin, parnaparin, certoparin, dalteparin, or tinzaparin.

In some specific embodiments, in the methods above as applicable, (a) the heparin (e.g., LMWH such as enoxaparin) is in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the heparin (e.g., LMWH such as enoxaparin) is in an amount of about 1 mg to about 200 mg (e.g., about 10 mg, about 50 mg, about 10) mg, or any range between the recited values); (b) the aliphatic acid of Formula I or pharmaceutically acceptable salt thereof is sodium caprate, an amount of about 0.6 mmol to 2 mmol (e.g., 0.6 mmol, 0.8 mmol, 0,9 mmol, 1 mmol, 1.1 mmol, 1.3 mmol, 2 mmol, or any range between the recited values, such as 0.9-1.1 mmol); and/or (c) the compound of Formula II or pharmaceutically acceptable salt thereof is SNAC, in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol).

In some specific embodiments, in the methods above as applicable, (a) the heparin is a low molecular weight heparin described herein, for example, in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 401) mg, or any range between the recited values).

In some specific embodiments, in the methods above as applicable, (a) the heparin is a low molecular weight heparin described herein, for example, in an amount of about 1 mg to about 200 mg (e.g., about 10 mg, about 50 mg, about 100 mg, or any range between the recited values); (b) the aliphatic acid of Formula I or pharmaceutically acceptable salt thereof is sodium caprate, in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and/or (c) the compound of Formula II or pharmaceutically acceptable salt thereof is SNAC, in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol).

In some specific embodiments, in the methods above as applicable, (a) the heparin is enoxaparin, for example, in an amount of about 1 mg to about 200 mg (e.g., about 10 mg, about 50 mg, about 100 mg, or any range between the recited values), or in an amount of about 1000 IU to :25000 IU; (b) the aliphatic acid of Formula I or pharmaceutically acceptable salt thereof is sodium caprate, in an amount of about 50 mg to about 300 mg (e.g., about 100 mg, about 150 mg, about 200 mg, or any range between the recited values); and/or (c) the compound of Formula II or pharmaceutically acceptable salt thereof is SNAC, in an amount of about 200 mg to about 400 mg (e.g., about 200 mg, about 300 mg, about 400 mg, or any range between the recited values).

In some specific embodiments, in the methods above as applicable, (a) the heparin is enoxaparin, for example, in an amount of about 1 mg to about 200 mg (e.g., about 10 mg, about 50 mg, about 100 mg, or any range between the recited values), or in an amount of about 1000 IU to 25000 IU; (b) the aliphatic acid of Formula I or pharmaceutically acceptable salt thereof is sodium caprate, in an amount of about 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.1 mmol); and/or (c) the compound of Formula II or pharmaceutically acceptable salt thereof is SNAC, in an amount of 0.6 mmol to 2 mmol (e.g., 0.6 mmol, 0.8 mmol, 0.9 mmol, 1 mmol, 1.1 mmol, 1.3 mmol, 2 mmol, or any range between the recited values, such as 0.9-1.3 mmol.

In some embodiments, the mixing in step (a) of the method herein comprises mixing with the optional chelating agent. For example, in some embodiments, the mixing in (a) comprises mixing (i), (ii), and ethylenediaminetetraacetic acid or a salt thereof, such as sodium salt.

In some embodiments, the mixing in step (a) of the method herein does not include mixing with the optional chelating agent. In some embodiments, the freeze-dried mixture formed in step (b) is further mixed with a chelating agent, such as ethylenediaminetetraacetic acid or a salt thereof, such as sodium salt. For example, in some embodiments, the freeze-dried mixture formed in step (b) can be compressed into a first layer, which is then mixed with the chelating agent. In some embodiments, the chelating, agent is compressed into a second layer, which can be combined such as compressed together with the first layer formed from the freeze-dried mixture formed in step (b). In some embodiments, more than one layer of the chelating agent can be combined, such as compressed together, with the first layer formed from the freeze-dried mixture formed in step (b).

When present in the pharmaceutical composition, the chelating agent is typically in an amount of about 10 mg to about 150 mg per unit dose. In some embodiments, all of the chelating agent is used in the mixing in step (a) of the method herein. For example, the freeze-dried mixture formed in step (b) contains all of the chelating agent. In some embodiments, at least a portion of the chelating agent is compressed into one or more layers, which is/are combined with a heparin-containing layer comprising the heparin, the aliphatic acid of Formula I or pharmaceutically acceptable salt thereof, and the compound of Formula II or pharmaceutically acceptable salt thereof. For example, in some embodiments, the method can include combining one layer comprising the chelating agent with the heparin-containing layer to form a bilayer structure, such as a bilayer tablet. In some embodiments, the method can include more than one layers comprising the chelating agent with the heparin-containing layer to form a multilayered structure, such as having the heparin-containing layer sandwiched by the layers comprising the chelating agent.

As discussed herein in the examples section, the freeze-drying process and/or having the chelating agent in one or more separate layers from the heparin-containing layer can be advantageous for example in providing enhanced oral delivery of heparin in some embodiments, in comparison to simple mixing and/or having heparin and the chelating agent included in the same layer. Thus, in some preferred embodiments, the method of preparing herein can preferably include a freeze-drying step. In some preferred embodiments, the pharmaceutical composition herein preferably includes one or more separate layers comprising the chelating agent.

The composition comprising the heparin prepared by the method herein is also a novel composition of the present disclosure.

In some embodiments, the present disclosure further provides a method of preparing a pharmaceutical composition comprising mixing the composition comprising the heparin prepared by the method herein with a pharmaceutically acceptable excipient (e.g., any of those described herein).

### Method of Treatment

The pharmaceutical compositions described herein can be useful for treating a disease or disorder in a subject in need thereof, wherein the disease or disorder can be any of those known to be treatable with heparins, such as any of the approved indications by the U.S. FDA for enoxaparin, dalteparin, and tinzaparin, see the approved labels as of the filing date of this application. The enhanced oral delivery of therapeutic agents as shown in the present disclosure can offer alternative and advantageous treatment options using heparins.

### Heparin's Anti-Thrombotic and Anti Embolytic Therapeutic Applications

Heparins are indicated for prophylaxis of ischemic complications in unstable angina and non-Q-wave mycordial infarction, prophylaxis of deep vein thrombosis (DVT) in patients following hip or knee replacement surgery, and prophylaxis of DVT in patients following abdominal surgery, and in patients at risk for thromboembolic complications. Heparins are also indicated for prophylaxis of DVT in medical patients with severely restricted mobility during acute illness, and treatment of DVT with or without pulmonary embolism (Enoxaparin FDA label insert). The initial treatment venous thromboembolism relies on is heparin and LMWH anticoagulants. These acute treatments lower risk and costs associated and reduce risk of recurrence. Anticoagulant treatment is effective in thinning the blood preventing clot growth. While heparin does not remove the clot, it is slowly dissolved over the course of about 3 months. LMWH can only be at reduced doses with careful observation on patients with renal failure since LMWH is cleared through the kidney.

For the initial treatment, if there is intermediate risk of pulmonary embolism, intravenous or subcutaneous UFH, or subcutaneous LMWH heparin is administered over the first 5 to 10 days (P. S. Wells, M. A. Forgie and M. A. Rodger: Treatment of venous thromboembolism. JAMA 311, 717-728 (2014)). The dosing is generally 170-200 IU/kg subcutaneously for LMWH and 230-300 IU/kg for UFH. These can be given as one dose or split into two smaller doses twice daily. After this initial period the patient can be transitioned to VKAs or a newer oral anticoagulant. This treatment normally continues for three months or longer to ensure that the risk has been reduced.

In high-risk situations where pulmonary embolism has been triggered by shock or hypertension the treatment parameters can be different. The initial treatment is an immediate intravenous bolus of UFH, then with thrombolytic therapy, surgical or catheter pulmonary embolectomy, followed by the same three month treatment used in lower risk patients. Special cases of thrombolic pulmonary embolism, such as in patients with cancer or pregnant, have to be treated differently. LMWHs are highly recommended in pregnant patients as they can be subcutaneously administered twice a day, forgoing the use of VKAs. The use of VKAs are contraindicated by pregnancy as there are side effects including teratogenic effects in the first trimester, and fetal or neonatal intracranial bleeding in the third trimester since VKAs can cross the blood brain barrier. In cancer patients the initial treatment time is extended 3-6 months with LMWH or VKAs but can continue indefinitely if the cancer is not cured since venous thromboembolism is 4-times more likely in cancer patients. If the cancer is cured or is in remission treatment is normally continued for 6 months (S. V. Konstantinides: 2014 ESC Guidelines on the diagnosis and management of acute. pulmonary embolism. European heart journal, 35, 3145-3146 (2014).

Ischemic complications of acute coronary syndrome, which includes myocardial infarction and unstable angina, can be prevented with heparin. Heparin is not the primary source of treatment and is normally used in conjunction with aspirin to inhibit platelet activation and prevent the growth of plaques or a vasodialator, like nitroglycerine, to widen the blood vessels. Both heparin and LMWH have been shown to reduce the recurrence rate of angina and myocardial infarction. Heparin also dampens the coagulation reactions to levels similar to patients with stable coronary artery disease. Similarly the thromboembolic complications associated with atrial fibrillation can also be treated and prevented with heparin, reducing the risk of stroke (A. Undas, K. Szuldrzyński, K. E. Brummel-Ziedins, W. Tracz, K. Zmudka and K. G. Mann: Systemic blood coagulation activation in acute coronary syndromes. Blood 113, 2070-2078 (2009).

### Heparins and Sickle Cell Disease (SCD)

Sickle cell disease is a monogenic disorder that afflicts approximately 100,000 Americans and millions of people worldwide (Mayo Clin Proc. December 2018; 93(12):1810-1824). It is characterized by hemolytic anemia, vaso-occlusive crises, relentless end-organ injury, and premature death. Vascular occlusion is responsible for much of the morbidity associated with sickle cell disease. Very limited approaches are available for the treatment of SCD or major symptoms associated with SCD. Currently, red blood cell transfusion and hydroxyurea are the major disease-modifying therapies available for SCD. Hematopoetic stem cell transplant is curative, but barriers to treatment are substantial and include a lack of suitable donors, immunologic transplant rejection, long-term adverse effects, prognostic uncertainty, and poor end-organ function, which is especially problematic for older patients.

Heparin pharmacological effects on SCD may stem from multiple mechanisms, including anti adhesion by blocking P-selectin, anti-coagulation, anti-oxidation, anti-inflammation (Mayo Clin Proc, 2018; 93(12): 18010-1824). Multiple heparins have been tested clinically in SCD patients with various degrees of evidence in alleviating the symptoms of SCD (Sevuparin, Dalteparin and Tinazaparin; Patrick Ellsworth, Jane A Little, Sevuparin trial for acute pain in sickle cell disease: the dog that did not bark, haematology Vol 8 May 2021; van Zuuren E J, Fedorowicz Z, Low-molecular-weight heparins for managing vaso-occlusive crises in people with sickle cell disease Cochrane Database of Systematic Reviews 2015, Issue 12).

### Heparins and COVID-19

Heparin is known to have anti-inflammatory effects both in the vasculature and in the airway, which could beneficially impact COVID-19-associated inflammation. The anti-inflammatory effects of heparin and its constituent heparan sulfate glycosaminoglycan fragments fall into two general mechanisms: 1) dampening of inflammation through interaction with proinflammatory proteins and 2) preventing adhesion and an influx of inflammatory cells to a diseased area. With respect to inflammatory cell infiltration, a phenomenon that has been observed pathologically in COVID-19 (N Engl J Med 2020; 383:120-128), heparin can directly interact with vascular endothelial cells leading to reduced recruitment of the innate immune system and direct inhibition of neutrophil activation. More broadly, heparin has been shown to dampen inflammation in other preclinical models characterized by robust inflammation including pancreatitis and sepsis. Clinically, the use of heparin as an anti-inflammatory agent has shown limited evidence of benefit in human diseases including inflammatory bowel disease, asthma, reactive airways disease, and acute coronary syndrome.

Coagulopathy and high rates of VTE in COVID-19 raise the possibility that heparin may benefit patient outcomes. The utility of heparin as an anticoagulant in COVID-19 was first posited by a retrospective report of 449 patients with COVID-19 from Wuhan, China, where prophylaxis in medical patients is relatively uncommon due to a low incidence of VTE (VTE risk profiles and prophylaxis in medical and surgical inpatients: the identification of Chinese hospitalized patients' risk profile for venous thromboembolism (DissolVE-2): a crosssectional study. Chest 155: 114-122, 2019.). Since these initial reports, there has been one study published and two studies available in preprint format that have investigated the effects of therapeutic heparin in COVID-19. One large study evaluating anticoagulation was an analysis of 2,773 patients with COVID-19 in the Mount Sinai Health System (Association of treatment dose anticoagulation with in-hospital survival among hospitalized patients with COVID-19. J Am Coll Cardiol 1097: 35218-35219, 2020.). In patients requiring invasive mechanical entilation included in this cohort, anticoagulation was associated with an in-hospital mortality of 29.1% compared with 62.7% for patients who did not receive anticoagulation. In contrast, this study found that patients who received anticoagulation were significantly more likely to require invasive mechanical ventilation. In a smaller retrospective cohort study evaluating 44 patients, those who received heparin had improved coagulation parameters and normalized immunity as evidenced by increased lymphocyte counts and decreased interleukin (IL)-6 levels compared with control subjects. Another group observed that initiation of heparin for 27 patients infected with COVID-19 improved oxygenation; this study did not include a control group (Heparin therapy improving hypoxia in COVID-19 patients: a case series (Preprint). medRxiv 2020.).

### Heparin Applications in Pregnancy Women

The use of anticoagulant therapy during pregnancy is challenging because of the potential for both fetal and maternal complications. The guidance: Antithrombotic Therapy and Prevention of Thrombosis, 9th ed: American College of Chest Physicians Evidence-Based Clinical Practice Guidelines focuses on the management of VTE and thrombophilia as well as the use of antithrombotic agents during pregnancy. The guidance recommends low-molecular-weight heparin for the prevention and treatment of VTE in pregnant women instead of unfractionated heparin (Grade 1B). For pregnant women with acute VTE, we suggest that anticoagulants be continued for at least 6 weeks postpartum (for a minimum duration of therapy of 3 months) compared with shorter durations of treatment (Grade 2C). For women who fulfill the laboratory criteria for antiphospholipid antibody (APLA) syndrome and meet the clinical APLA criteria based on a history of three or more pregnancy losses, we recommend antepartum administration of prophylactic or intermediate-dose unfractionated heparin or prophylactic low-molecular-weight heparin combined with low-dose aspirin (75-100 mg/d) over no treatment (Grade 1B). For women with inherited thrombophilia and a history of pregnancy complications, we suggest not to use antithrombotic prophylaxis (Grade 2C). For women with two or more miscarriages but without APLA or thrombophilia, we recommend against antithrombotic prophylaxis (Grade 1B).

Antiphospholipid Syndrome (APS) has been widely recognized as a risk factor for the recurrence of both thrombosis and pregnancy losses however the optimal treatment of patients is debatable. The aim of this paper was to establish a consensus among experts on the treatment of APS in pregnancy, A questionnaire that described possible different clinical situations was sent to the International Advisory Board of the 10th international Congress on Antiphospholipid Antibodies. Sixteen experts from different medical branches and different geographic areas sent their replies. The consensus was that treatment for APS pregnant patients should be low molecular weight heparin (LMWH) and low dose aspirin (LDA). The dosage, and frequency of LMWH depends on different situations, including the body weight and past history.

### Oral Administration of Heparins

As discussed above, presently, heparin is administered parenterally, either by continuous or intermittent intravenous infusion or by deep subcutaneous injection.

Oral administration of hydrophilic macromolecules with a molecular weight (MW) above 1000 Da remains a challenge due to susceptibility to pH and gastric/small intestinal enzymes, as well as low intestinal epithelial membrane permeability. The low permeability results from minimal passive or carrier-mediated transcellular permeation across phospholipid bilayers, as well as restricted paracellular transport via tight junctions. Investigators attempted to address pre-systemic degradation and poor permeation in the same formulation. A common approach is to combine peptidase inhibitors with absorption-modifying excipients or chemical permeation enhancers (PEs), in addition to avoiding degradation by gastric enzymes and low pH, enteric-coated capsules and tablets avoid dilution and premature release of both PE and macromolecule in the stomach.

Oral delivery of unfractionated heparin has been investigated and evaluated in clinical trials, initial clinical studies with oral heparin-SNAC showed that: a) the safety and tolerability of unformulated solutions of heparin/SNAC combination b) that heparin without SNAC administered orally is not absorbed, c) that SNAC by itself has no effects on coagulation and d) to establish the minimal dose of oral heparin required to have a pharmacological effect when combined with maximal safe dose of SNAC. A single dose of 30,000 USP Heparin Units or SNAC alone at doses ranging from 1.4 to 10.5 g were administered orally. Measurements of anticoagulation (aPTT, TFPI and anti-Factors IIa and Xa levels) did not change following administration of either Heparin alone or SNAC alone. In a subsequent study, an unformulated solution of SNAC (10.5 g) in combination with 10,000, 20,000 or 30,000 USP heparin units were administered orally. Elevated aPTT, anti-Factors IIa and Xa levels were observed following oral administration of 20,000 Units and 30,000 Units in combination with 10.5 g of SNAC, demonstrating that SNAC facilitates the absorption of heparin when administered as heparin/SNAC solution in human subjects [13]. A taste-masked SNAC/Heparin oral solution formulation was developed and evaluated at a fixed dose (2.25 g) of SNAC in combination with escalating doses (30,000,60,000, 90,000 and 150,000 Units) of heparin. In a phase II clinical trial 123 patients undergoing total hip replacement were randomized to receive either or a heparin/SNAC (1.5 g SNAC/60.000 units UFH or 2:25 g SNAC/90.000 units UFH) as a taste masked oral solution or 5000 Units UFH s·c every 8 hours. Patients received study medication for 5 days and were observed for 35 days following surgery. An international, multi-center phase III thromboprophylaxis trial in 2264 patients with the objective to compares safety and efficacy of two oral doses of UFH to a standard subcutaneous LMWH regimen (PROTECT trial) inpatients undergoing elective hip surgery was conducted. The clinical trial was randomized, double-blind (double dummy: placebo oral or injection). Oral heparin prophylaxis was initiated 4-6 hours postoperatively and continued through the whole evaluation period (27-30 days), while enoxaparin was initiated 12-24 hours postoperatively and was administered for 10 days followed by placebo until the final evaluation. Oral heparin/SNAC solution, low dose 60,000 IU/1.5 g SNAC (IdSNAC) and high dose 90,000 IU/2.25 g SNAC (hdSNAC) were administered trice daily. The results from the PROTECT trial made clear the need to overcome the adverse taste of the oral heparin formulation and attain a more patient-friendly formulation of heparin/SNAC with the focus being a solid dosage form.

A more efficient and friendly solid formulation than the liquid formulation is important in achieving sufficient and less variable exposure, improving clinical compliance, and easiness in manufacturing/storage/distribution, thus reducing overall cost of the oral products. Furthermore, LMWH has proven to be more favorable for its longer T1/2, safer profile without the need of monitoring anti-coagulation activity, there is also a need of innovations in oral forms of LMWH.

As shown in details herein, it was found that the pharmaceutical compositions herein any of those according to [1]-[37] and [55]-[57] as described in the Brief Summary Section, as applicable) can be used to deliver an effective amount of heparin, such as LMWH, in particular, enoxaparin, following oral administration.

In some embodiments, the present disclosure provides a method of treating a disease or disorder for which administering heparin such as LMWH is beneficial, e.g., deep vein thrombosis (DVT), Sickle Cell Disease (SCD), pre-thrombotic state/recurrent spontaneous abortion (PTS/RSA), cancer associated thrombosis (CAT), dyslipidemia, etc., in a subject in need thereof, the method comprising orally administering the pharmaceutical composition of any of those described herein to deliver a therapeutically effective amount of the heparin such as LMWH to the subject. In some embodiments, the present disclosure provides a method of prophylaxis or treatment of COVID-19 infection in a subject in need thereof, the method comprising orally administering the pharmaceutical composition of any of those described herein to deliver an effective amount of the heparin such as LMWH to the subject. In some embodiments, the pharmaceutical composition can be any of those described according to [1]-[37] and [55]-[57] as described in the Brief Summary Section, as applicable.

In some specific embodiments, the present disclosure provides a method of treating or preventing sickle cell disease (SCD) in a human in need thereof, the method comprising orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA.

In some specific embodiments, the present disclosure provides a method of treating or preventing pre-thrombotic state/recurrent spontaneous abortion (PTS/RSA)in a human in need thereof, the method comprising orally administering an effective amount of one or more pharmaceutical compositions described herein (e.g., any of those according to [1]-[37] and [55]-[57] as described in the Brief Summary Section, as applicable), such as those comprising LMWH sodium caprate, SNAC, and optionally EDTA.

In some specific embodiments, the present disclosure provides a method of treating or preventing cancer associated thrombosis (CAT) in a human in need thereof, the method comprising orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA.

In some specific embodiments, the present disclosure provides a method of prophylaxis or treatment of COVID-9 infection in a human in need thereof, the method comprising orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA.

In some specific embodiments, the present disclosure provides a method of treating or preventing dyslipidemia in a human in need thereof, the method comprising orally administering an effective amount of one or snore pharmaceutical compositions described herein (e.g., any of those according to [1]-[37] and [55]-[57] as described in the Brief Summary Section, as applicable), such as those comprising LMWH, sodium caprate, SNAC, and optionally EDTA.

In some specific embodiments, the present disclosure provides a method of treating or preventing thrombosis in a human in need thereof, the method comprising orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA. Any type of thrombosis can be treated or prevented with the pharmaceutical composition including, but not limited to, deep vein thrombosis (DVT) and pulmonary embolism (PE).

In some specific embodiments, the present disclosure provides a method of treating or preventing deep vein thrombosis in a human in need thereof, the method comprising orally administering an effective amount of one or more pharmaceutical compositions described herein (e.g., any of those according to [1]-[37] and [55]-[57] as described in the Brief Summary Section, as applicable), such as those comprising sodium caprate, SNAC, and optionally EDTA.

In some specific embodiments, the present disclosure provides a method of treating or preventing ischemic complications in unstable angina and/or non-Q-wave mycordial infarction in a subject in need thereof by orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA.

In some specific embodiments, the present disclosure provides a method of treating or preventing deep vein thrombosis (DVT) in a subject following hip or knee replacement surgery in a subject in need thereof by orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA.

In some specific embodiments, the present disclosure provides a method of treating or preventing DVT following abdominal surgery in patients at risk for thromboembolic complications in a subject in need thereof by orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA. Abdominal surgery patients at risk include, but are not limited to, those who are over 40 years of age, obese, undergoing surgery under general anesthesia lasting longer than 30 minutes or who have additional risk factors such as malignancy or a history of DVT or pulmonary embolism.

In some specific embodiments, the present disclosure provides a method of treating or preventing DVT in a subject with severely restricted mobility in a subject in need thereof by orally administering an effective amount of one or more pharmaceutical compositions described herein (e.g., any of those according to [1]-[37] and [55]-[57] as described in the Brief Summary Section, as applicable), such as those comprising LMWH, sodium caprate, SNAC, and optionally EDTA.

The pharmaceutical compositions herein (e.g., any of those according to [1]-[37] and [55]-[57] as described in the Brief Summary Section, as applicable) can also be used for additional treatment methods which include, but are not limited to the treatment of cardiac valve replacements, both mechanical and cadaver; treatment of endocarditis; prophylaxis in patients undergoing neurological procedures, such as, but not limited to, resection of malignant brain tumors; prophylaxis in patients with acute spinal cord injury, medical conditions associated with thromboembolism, such as but not limited to, those with ischemic stroke or restricted mobility, cancer, myocardial infarction, cancer, congestive heart failure, or severe pulmonary disease; for secondary prophylaxis of venous thromboembolism during pregnancy, or for primary prophylaxis in pregnant women with inherited causes of thrombophilia (e.g., deficiencies in thrombin III, protein C, protein S, Factor V, Leiden mutation, prothrombin polymorphism, hyperhomocysteinemia); for embolisms associated with atrial fibrillation or those with concurrent prosthetic valves and atrial fibrillation; for cardioversion of atrial fibrillation or atrial flutter; for disseminated intravascular coagulation; to reduce the risk of complications in patients undergoing percutaneous coronary intervention, percutaneous transluminal coronary angioplasty, arthrectomy, coronary or other vessel stent implantation, ischemic cerbrovascular accident, hemodialysis, peripheral vascular interventions, acute myocardial infarction; unstable angina and non-ST-Segment Elevation Myocardial infarction; cerebral thromboembolism; complications of pregnancy, including but not limited to pregnancy loss in women with a history of antiphospholipid antibodies/antiphospholipid syndrome with or without fetal loss, fetal death or fetal miscarriage. Other uses of heparin may be found in Drug Information, American Society of Health System Pharmacists, 2005, which is incorporated herein by reference.

### Definitions

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone), Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

The term solid dosage form can refer to a tablet, or a capsule filled with solids, or a capsule filled with a solution.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of a therapeutic agent (e.g., LMWH) sufficient to result in amelioration of one or more symptoms of a disorder or condition (e.g., DVT, sickle cell disease (SCD), etc.), or prevent appearance or advancement of a disorder or condition, or cause regression of or cure from the disorder or condition.

The term “subject” (alternatively referred to herein as “patient”) as used herein, refers to an animal, preferably a mammal, most preferably a human, who has been the object of treatment, observation or experiment.

### EXAMPLES

### Example 1

This example shows representative procedures of preparing oral formulations containing enoxaparin.

### Preparation of Formulations

The preparation of oral tablet enoxaparin formulation includes generally three steps: mixing, compressing, and optionally for enteric coated tablets, also the coating step. Mixing can be simple blending or involve freeze drying.

Simple blending: Weigh the enoxaparin, SNAC, sodium caprate (C10) (and other excipients if necessary) based on the formulation into a mortar. Use the pestle to grind the mixer at least 30 minutes to make sure they could mix well.

Freezing drying: Weigh enoxaparin and add it into a glass vial. Add ˜15 ml of water to dissolve each 500 mg of enoxaparin. Then weigh SNAC and add it into the enoxaparin above mentioned water solution. Use a stir bar for stirring. At last, add C10 (and other excipients, if necessary), into the solution. Stir the solution to make all excipients dissolved. Put the solution into −20° C. freezer for a period of about 2 h. Then transfer the solution to −80° C. freezer for prefreezing overnight. After prefreezing, put the glass vials into freezing dryer for drying using the following protocol.

Compressing: After mixing, weigh appropriate amount of the mixer, and use a manual compressor to compress the mixer to a tablet. The mold is a round tablet mold with a diameter of 12 mm, and with an arc in the surface of the mold. Compress the mold hard by our arms 9˜12 times, then a tablet will be obtained.

Coating: Caleva Tablet and Pellet Mini Coater Drier (MCD-2) was used for enteric coated tablets. Eudragit L³⁰D-55 was used as the enteric coating polymer. The Eudragit was prepared to a 20% solid solution containing 2% of triethyl citrate (TEC) and 10% talc. The coating parameters are as followed: Agitator: 12˜15 Hz; Fan: 13.0˜15.2 m/sec; Heat: set at 60° C.; Pump: 1.09˜1.48 RPM; Air: 22˜30 psi. After weight gain of the tablet reach to 10% or 20%, stop coating.

Following the general procedures above, the following formulations were prepared without the enteric coating step:

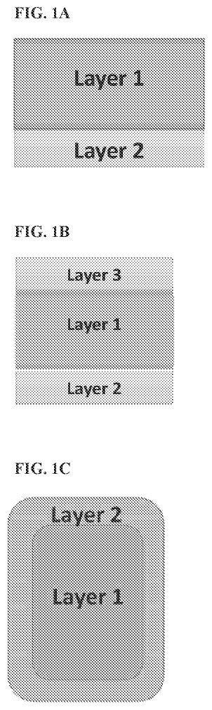

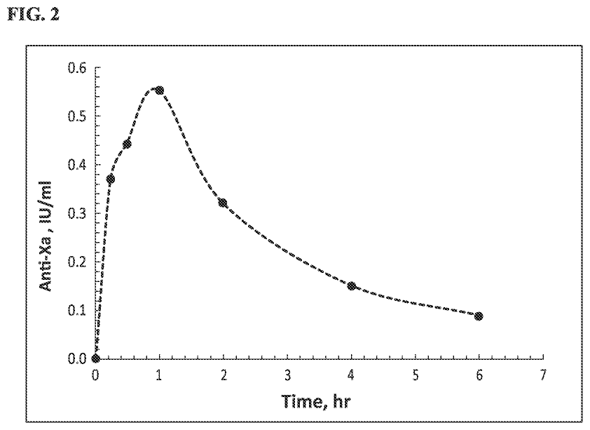

Formulation 1 (EX1): which is a two layer formulation, see FIG. 1A, with the following ingredients: Layer 1: 300 mg SNAC+150 mg C10+80 mg Enoxaparin; and Layer 2: 50 mg EDTA-Na₂·2H₂O. For preparation of Layer 1, the mixing method was based on dissolving all components and freeze-drying. Layer 1 and Layer 2 were compressed together.

Formulation 2 (EX2): which is a two layer formulation, see FIG. 1A, with the following ingredients: Layer 1: 300 mg SNAC+150 mg C10+80 mg Enaxoparin; and Layer 2: 50 mg EDTA-Na₂·2H₂O, For preparation of Layer 1, the mixing method was based on simple blending of all components in their solid forms.

Formulation 3A (EX3A): which is a one layer formulation, having the following ingredients: 300 mg SNAC+150 mg C10+80 mg Enaxoparin+50 mg EDTA-Na₂·2H₂O. Mixing method: simple blending of ingredients.

Formulation 3B (EX3B): which is a one layer formulation, having the following ingredients: 300 mg SNAC+150 mg C10+80 mg Enaxoparin+50 mg EDTA-Na₂·2H₂O. Mixing method: Dissolving and Freeze-drying all components together in one structure.

Formulation 4 (EX4): which is a three layer formulation, see FIG. 1B, with the following ingredients: Layer 1: 300 mg SNAC+150 mg C10+80 mg Enoxaparin; Layer 2: 37.5 mg EDTA-Na₂·2H₂O; and Layer 3: 37.5 mg EDTA-Na₂·2H₂O. For preparation of Layer 1, the mixing method was based on dissolving all components and freeze-drying, Layers 1, 2, and 3 were compressed together.

Formulation 5 (EX5): which is a one layer formulation, having the following ingredients: Enoxaparin 80 mg+SNAC 300 mg+C10+150 mg . Mixing method: Dissolving and Freeze-drying all components and compressing into one layer.

Formulation 6A (EX6A): which is a one layer formulation, having the following ingredients: Enoxaparin 80 mg+SNAC 300 mg+C10+150 mg . Mixing method: Dissolving and Freeze-drying all components and compressing into one layer. Formulation 6A is the same as Formulation 5, but was tested along with Formulation 6B and 6C for comparison.

Formulation 6B (EX6B): which is a one layer formulation, having the following ingredients: Enoxaparin 80 mg+SNAC 450 mg. Mixing method: Dissolving and Freeze-drying all components and compressing into one layer.

Formulation 6C (EX6C): which is a one layer formulation, having the following ingredients: Enoxaparin 80 mg+SNAC 300 mg. Mixing method: Dissolving and Freeze-drying all components and compressing into one layer.

Formulation 7 (EX7): which is a one layer formulation, having the following ingredients: Enoxaparin 80 mg+C10+300 mg . Mixing method: Dissolving and. Freeze-drying all components and compressing into one layer.

Formulation 8A (EX8A): which is a one layer formulation, having the following ingredients: Enoxaparin 80mg+SNAC 300 mg+C10+150 mg. Mixing method: Dissolving and Freeze-drying all components and compressing into one layer. Formulation 8A is the same as Formulation 5, but was tested along with Formulation 8B and 8C for comparison,

Formulation 8B (EX8B): which is a one layer formulation, having the following ingredients: Enoxaparin 80 mg+SNAC 150 mg+C10 150 mg. Mixing method: Dissolving and -Freeze-drying all components and compressing into one layer.

Formulation 8C (EX8C): which is a one layer formulation, having the following ingredients: Enoxaparin 80 mg+SNAC 1.50 mg+C10 300 mg. Mixing method: Dissolving and Freeze-drying all components and compressing into one layer.

Formulation 9A (EX9A): which is a one layer formulation, having the following ingredients: Enoxaparin 80 mg 300 mg SNAC+150 mg C10+50 mg EDTA-Na₂·2H₂O. Mixing method: Dissolving and Freeze-drying all components and compressing into one layer.

Formulation 9B (EX9B): which is a one layer formulation, having the following ingredients: Enoxaparin 80 mg+300 mg SNAC+150 mg C10+100 mg EDTA-Na₂·2H₂O. Mixing method: Dissolving and Freeze-drying all components and compressing into one layer.

Formulation 9C (EX9C): which is a one layer formulation, having the following ingredients: Enoxaparin 80mg+300 mg SNAC+150 mg+C₁₀₊₂₀₀ mg EDTA-Na₂·2H₂O. Mixing method: Dissolving and Freeze-drying all components and compressing into one layer.

Sc injection: which is an injected sodium enoxaparin solution with 80 mg/ml, by dissolving 80 mg of sodium enoxaparin into 1 ml of ultrapure water and then filtering it with a 0.22 μm membrane filter. Each animal was dosed 0.1 ml , equal to 8 mg of sodium enoxaparin, in the subcutaneous layer of the neck skin.

### Example 2. Pharmacokinetics Studies

This example describes pharmacokinetics (“PK”) studies

Dogs weighing approximately 11-12 Kg were raised in separated cages. Before an experiment, two meals were supplied to the dogs everyday at about 3 p.m. and 7 a.m. The foods are fodder with water in a bowl. In the day before an experiment, after feeding the dogs at 3 p.m., the dogs were fasted overnight and throughout the whole experimental period. Water was freely provided for the dogs overnight, but was removed at 1 h before the experiment. Before dosing, the dogs were put into a sling and then a 0.5 ml blood sample was collected from each dog's leg vein using a sterile disposable syringe. For dosing, two dosing methods are described below.

Tablet oral dosing: open the cages and let the dogs climb on the cage by their forelegs. Catch the mouth and open the mouth by hand, Workers will keep their mouth open and put the tablet into dog's throat using their fingers. Then close the dog's mouth and keep the mouth closed for about ten seconds. The dogs would swallow the tablet naturally without any water.

sc injection: Pinch some loose skin from the back of the neck region between thumb and forefinger. Insert the needle swiftly into the fold of the skin, with the needle angled downwards at a fifteen to twenty degree angle. Administer the content of the syringe quickly and withdraw the needle.

After dosing, 0.3 ml of blood samples were collected at time points of 15 min, 30 min, 1 h, 2 h, 4 h, and 6 h from each dog's foreleg vein. All the bloods are collected into EP tubes with 0.109 M citric acid solution as anticoagulant. The volume ratio of blood with citric acid is v/v (9:1). After turning the tubes upside down for several times for mixing, the blood samples were centrifuged at 3500 rpm for ten minutes. The supernatant plasmas were removed to clean EP tubes and stored at −20° C. freezer before measurements. After collecting the 6 h time point samples, the dogs were released, and food and water were returned to the dogs immediately.

Dogs were put into a sling to restrict their movement. Before dosing, 0.5 ml of blood was collected. After dosing, 0.3 ml blood samples were collected at timepoints of 15 min, 30 min, 1 h, 2 h, 4 h, and 6 h from each dog's foreleg vein. All the blood samples are collected into EP tubes with 0.109 M citric acid solution as anticoagulant as v/v (9:1). After turning the tubes upside down several times for mixing, the blood samples were centrifuged at 3500 rpm for ten minutes. The supernatant plasmas were removed and stored at −20° C. freezer before measurements.

### Anti-Xa Assay Method

The BIOPHEN™ Anti-Xa (2 stages Heparin Assay) kit is a chromogenic anti-Xa method, developed for measuring unfractionated heparin and low molecular weight heparins in plasma or in purified solutions,
Heparin+AT→[AT Hep.]
[AT Hep.]+[FXa (excess)]→[FXa-AT-Hep.]+[residual FXa]
[residual FXa]+substrate→peptide+pNA
R1 reagent: ATIII
R2 reagent: FXa
R3 reagent Factor Xa specific chromogenic substrate

The detailed procedures are the following:
1. Thaw the reagents as rapidly as possible at 37° C.;
2. Add 1 ml of water into R1, R2, R3 to dissolve the solid reagents. Shake vigorously until fully dissolved. Allow to stabilize for 30 min at room temperature (18-25° C.):
3. Add 4 ml of buffer to dilute the R1, R2, R3 reagent. (R1, R2: buffer AR005L (Tris-NaCl buffer, pH 7.40); R3: AR029K (Tris EDTA NaCl buffer, pH 8.40));
4. Dissolve the enoxaparin standard in water and then use placebo plasma to progressively dilute it to obtain eight concentrations of standard solutions (0˜1.2 IU/ml).
5. Use 140 μl AR005L buffer to dilute 10 μl of samples (or standard solutions). Then transfer 40 μl of the diluted samples into each well of the 96-well plates. The eight standard solutions were set at the first row and the fifth row. Samples were set at row 2 to row 4. No more than 5 rows of samples were measured at one time to ensure an accurate reaction time.
6. Pre-warm the samples in the 96-well plates at a specific incubator for 5 minutes. R1 reagent, R2 reagent, and R3 reagent were also pre-warmed at 37° C. shaker for 15 minutes.
7. Open the incubator and the cover of the 96-well plate, use a twelve channel pipette to add 40 μl of R1 into the samples row by row. Record the time after adding R1 into first row. After adding the R1, return the cover to 96-well plate and close the incubator. React them 2 minutes accurately at 37° C. and with an agitation speed of 400 rpm.
8. After 2 minutes, add 40 μl of R2 into the samples by same procedure as above and react them 2 minutes accurately;
9. After 2 minutes, add 40 μl of R3 into the samples by same procedure as above and react them 2 minutes accurately;
10. After 2 minutes, add 80 μl of 2% citric acid to stop the reaction. Read the color intensity at 405 nm on an ELISA.
11. Average the two rows of standards and use binomial fitting to fit the standard curve. Then calculate the anti-Xa level of the samples.

### PK Results

The PK results obtained for Formulation 1 are shown in FIG. 2, and also shown in Table 1 below:

TABLE 1
The PK data for Formulation 1.

| Time | Mean | CV % |
|---|---|---|
| 15 min | 0.372 | 72 |
| 30 min | 0.444 | 79 |
| 1 h | 0.555 | 82 |
| 2 h | 0.323 | 94 |
| 4 h | 0.153 | 59 |
| 6 h | 0.091 | 70 |

The PK results obtained for Formulation 2 are shown in Table 2 below:

TABLE 2
The PK data for Formulation 2.

| Time | Mean | CV % |
|---|---|---|
| 15 min | 0.14 | 86 |
| 30 min | 0.273 | 60 |
| 1 h | 0.225 | 66 |
| 2 h | 0.202 | 139 |
| 4 h | 0.058 | 103 |
| 6 h | 0.049 | 86 |

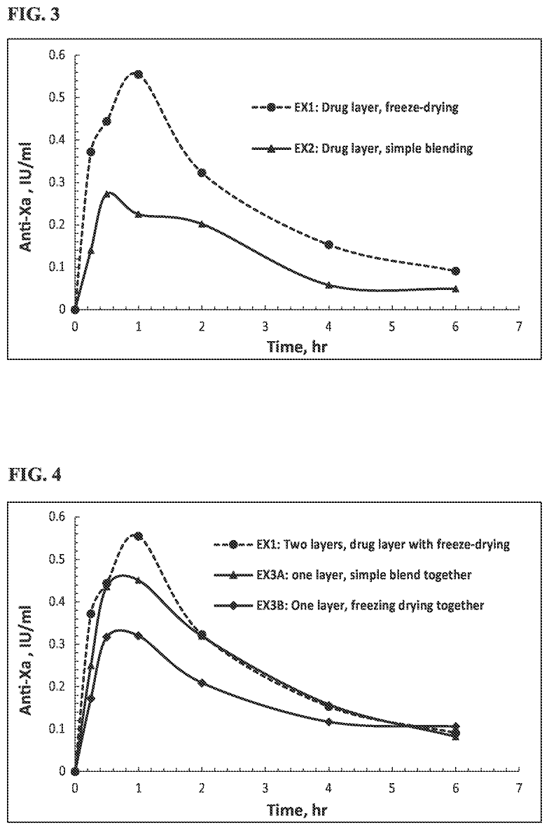

A comparison of PK obtained for Formulation 1 and Formulation 2 is shown in FIG. 3, and also shown in Table 3 below:

TABLE 3
Comparison of the AUClast and Cmax between
Formulation 1 and Formulation 2.

| | Formulation 1 | Formulation 2 |
|---|---|---|
| Cmax (CV %) | 0.555 (82) | 0.273 (60) |
| AUClast (CV %) | 1.561 (69) | 0.777 (98) |

The results show that Formulation 1 achieved increased AFXa activity compared to administering Formulation 2, indicating that freeze drying of carriers and drug is beneficial.

The PK results obtained for Formulations 3A and 3B are shown in Table 4 below.

TABLE 4
The PK data for Formulation 3A (EX3A)
and Formulation 3B (EX3B).

| | EX3A: simple blend together | | EX3B: freeze-drying together | |
|---|---|---|---|---|
| Time | Mean | CV % | Mean | CV % |
| 15 min | 0.25 | 123 | 0.172 | 85 |
| 30 min | 0.436 | 107 | 0.317 | 81 |
| 1 h | 0.451 | 109 | 0.320 | 80 |
| 2 h | 0.319 | 125 | 0.209 | 91 |
| 4 h | 0.158 | 119 | 0.117 | 94 |
| 6 h | 0.082 | 118 | 0.106 | 101 |

A comparison of PK obtained for Formulation 1 and Formulations 3A and 3B is shown in FIG. 4, and also shown in Table 5 below:

TABLE 5
Comparison of the AUClast and Cmax between Formulation
1 (EX1) and Formulations 3A and 3B (EX3A and 3B).

| | EX1 | EX3A | EX3B |
|---|---|---|---|
| Cmax (CV %) | 0.555 (82) | 0.451 (109) | 0.320 (80) |
| AUClast (CV %) | 1.561 (69) | 1.443 (113) | 1.057 (88) |

The above data show that better PK results were obtained from Formulation 1 compared to Formulation 3A or 3B, indicating formulating EDTA into a separate layer is beneficial.

The PK results obtained for Formulation 4 are shown in Table 6 below.

TABLE 6
The PK data for Formulation 4.

| Time | Mean | CV % |
|---|---|---|
| 15 min | 0.260 | 67 |
| 30 min | 0.430 | 56 |
| 1 h | 0.413 | 65 |
| 2 h | 0.197 | 61 |
| 4 h | 0.151 | 71 |
| 6 h | 0.093 | 43 |

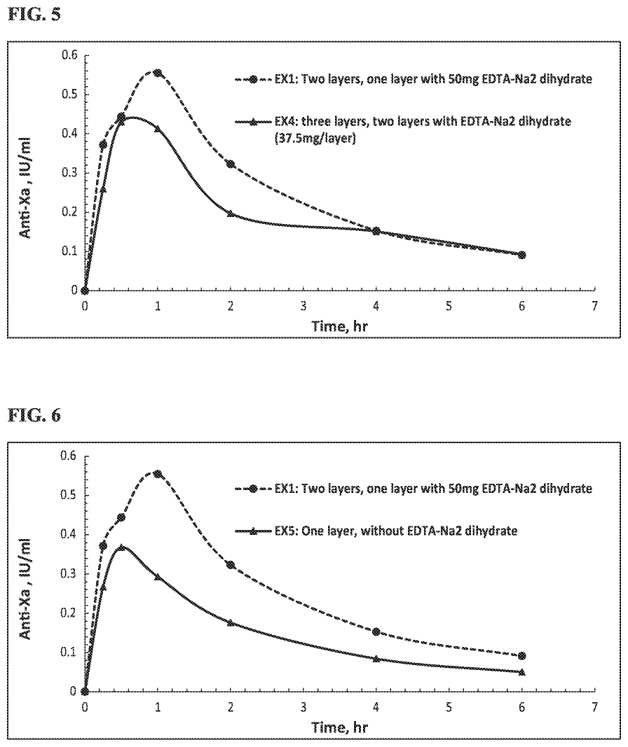

A comparison of PK obtained for Formulation 1 and Formulation 4 is shown in FIG. 5, and also shown in Table 7 below:

TABLE 7
Comparison of the AUClast and Cmax between Formulation
1 (EX1) and Formulation 4 (EX4).

| | EX1 | EX4 |
|---|---|---|
| Cmax (CV %) | 0.555 (82) | 0.430 (56) |
| AUClast (CV %) | 1.561 (69) | 1.230 (57) |

The above data show that the three-layer structure with the middle layer as SNAC/C10/Enoxaparin, and outer two layers as EDTA also achieved satisfactory results. It is expected that a surrounded two-layer structure, see FIG. 1C, will provide similar results.

The PK results obtained for Formulation S are shown in Table 8 below.

TABLE 8
The PK data for Formulation 5 (EX5).

| Time | Mean | CV % |
|---|---|---|
| 15 min | 0.267 | 83 |
| 30 min | 0.368 | 87 |
| 1 h | 0.293 | 88 |
| 2 h | 0.176 | 99 |
| 4 h | 0.084 | 86 |
| 6 h | 0.05 | 83 |

A comparison of PK obtained for Formulation 1 and Formulation 5 is shown in FIG. 6, and also shown in Table 9 below:

TABLE 9
Comparison of the AUClast and Cmax between Formulation
1 (EX1) and Formulation 5 (EX5).

| | EX1 | EX5 |
|---|---|---|
| Cmax (CV %) | 0.555 (82) | 0.368 (87) |
| AUClast (CV %) | 1.561 (69) | 0.910 (89) |

The results show that adding a separate EDTA layer to SNAC/C10/Enoxaparin layer based on freeze-drying is superior to the one layer structure with dissolving, freeze-drying and compressing all components together

The PK results obtained for Formulations 6A, 6B, and 6C are shown in Table 10 below.

TABLE 10
The PK data for studies of addition C10 and without C10.

| | | SNAC 300 mg, addition | | SNAC 300 mg | | SNAC 450 mg | |
|---|---|---|---|---|---|---|---|
| | | C10 150 mg (6A) | | (6C) | | (6B) | |
| Time | | Mean | CV % | Mean | CV % | Mean | CV % |
| 15 | min | 0.27 | 51 | 0.009 | 439 | −0.003 | −553 |
| 30 | min | 0.565 | 9 | 0.125 | 46 | 0.045 | 245 |
| 1 | h | 0.645 | 20 | 0.172 | 70 | 0.099 | 79 |
| 2 | h | 0.365 | 9 | 0.098 | 64 | 0.145 | 122 |
| 4 | h | 0.146 | 6 | 0.067 | 45 | −0.018 | −123 |
| 6 | h | 0.076 | 24 | 0.042 | 65 | −0.041 | −76 |

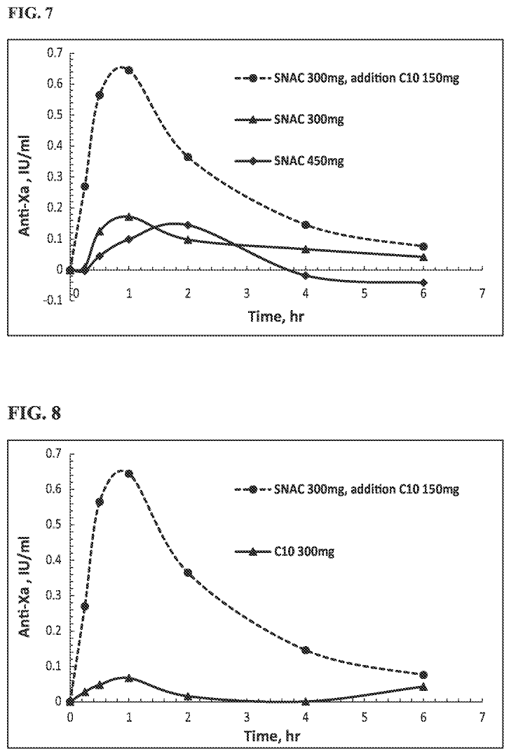

A comparison of PK obtained for Formulations 6A, 6B, and 6C is shown in FIG. 7, and also shown in Table 11. below:

TABLE 11
Comparison of the AUClast and Cmax between
addition C10 and without C10.

| | SNAC 300 mg, addition | SNAC | SNAC |
|---|---|---|---|
| | C10 150 mg | 300 mg | 450 mg |
| | (6A) | (6C) | (6B) |
| Cmax (CV %) | 0.645 (20) | 0.172 (70) | 0.145 (122) |
| AUClast (CV %) | 1.682 (11) | 0.502 (55) | 0.229 (70) |

The above results show that SNAC 300 mg+C10 150 mg achieved surprising synergy. Either increasing SNAC 300 to 450 mg, or with SNAC 300 mg alone showed much lowed exposure.

The PK results obtained for Formulation 7 are shown in Table 12 below.

TABLE 12
The PK data for studies of combination
SNAC with C10 and C10 only

| SNAC 300 mg, addition | |
|---|---|
| C10 150 mg | C10 300 mg |
| (EX6A) | (EX7) |

| Time | Mean | CV % | Mean | CV % |
|---|---|---|---|---|
| 15 min | 0.27 | 51 | 0.028 | 164 |
| 30 min | 0.565 | 9 | 0.048 | 241 |
| 1 h | 0.645 | 20 | 0.067 | 70 |
| 2 h | 0.365 | 9 | 0.016 | 204 |
| 4 h | 0.146 | 6 | 0.001 | 1614 |
| 6 h | 0.076 | 24 | 0.043 | 145 |

A comparison of PK obtained for Formulations 6A (EX6A) and 7 (EX7) is shown in FIG. 8, and also shown in Table 13 below:

TABLE 13
Comparison of the AUClast and Cmax between EX6A
(combination SNAC with C10) and EX7 (C10 only).

| | SNAC 300 mg, addition | |
|---|---|---|
| | C10 150 mg | C10 300 mg |
| | (EX6A) | (EX7) |
| Cmax (CV %) | 0.645 (20) | 0.067 (70) |
| AUClast (CV %) | 1.682 (11) | 0.145 (161) |

The results show that C10 alone did not achieve a satisfactory exposure. Combining the results from SNAC alone further supports the synergistic effect of SNAC and C10 in enhancing oral PK profile of enoxaparin.

The PK results obtained for Formulations 8A, 8B, and 8C are shown in Table 14 below.

TABLE 14
The PK data for studies of different SNAC/C10 ratio

| | | SNAC/C10 = 2:1 | | SNAC/C10 = 1:1 | | SNAC/C10 = 1:2 | |
|---|---|---|---|---|---|---|---|
| | | (8A) | | (8B) | | (8C) | |
| Time | | Mean | CV % | Mean | CV % | Mean | CV % |
| 15 | min | 0.28 | 62 | 0.341 | 80 | 0.262 | 96 |
| 30 | min | 0.458 | 61 | 0.328 | 73 | 0.358 | 108 |
| 1 | h | 0.388 | 64 | 0.251 | 85 | 0.338 | 101 |
| 2 | h | 0.25 | 64 | 0.159 | 84 | 0.227 | 90 |
| 4 | h | 0.115 | 60 | 0.117 | 129 | 0.104 | 123 |
| 6 | h | 0.086 | 56 | 0.046 | 112 | 0.082 | 107 |

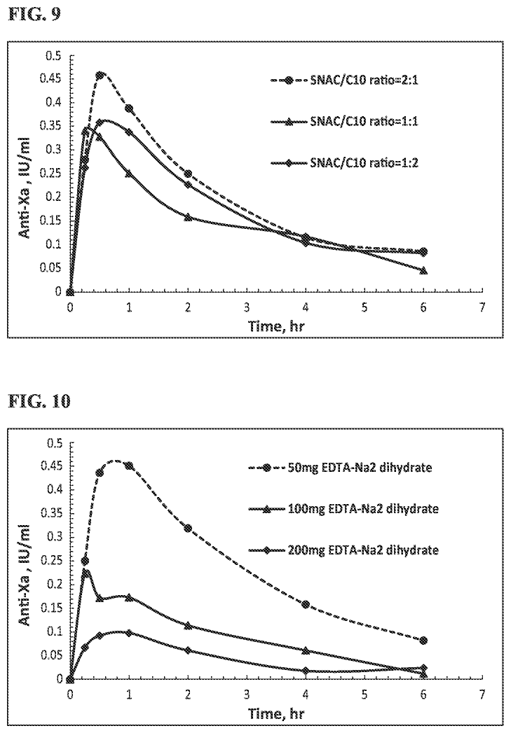

A comparison of PK obtained for Formulations 8A, 8B, and 8C is shown in FIG. 9, and also shown in Table 1.5 below:

TABLE 15
Comparison of the AUClast and Cmax
between different SNAC/C10 ratio.

| | SNAC/C10 = | SNAC/C10 = | SNAC/C10 = |
|---|---|---|---|
| | 2:1 | 1:1 | 1:2 |
| | (8A) | (8B) | (8C) |
| Cmax (CV %) | 0.458 (61) | 0.341 (80) | 0.358 (108) |
| AUClast (CV %) | 1.227 (60) | 0.918 (94) | 1.086 (103) |

The results above show that different ratios of SNAC and CIO are effective, with 300 mg SNAC+150 mg+C10 more preferable.

The PK results obtained for Formulations 9A, 9B, and 9C are shown in Table 16 below.

TABLE 16
The PK data for studies of different EDTA amount

| | | 50 mg EDTA- | | 100 mg EDTA- | | 200 mg EDTA- | |
|---|---|---|---|---|---|---|---|
| | | Na₂•2H₂O | | Na₂•2H₂O | | Na₂•2H₂O | |
| | | (9A) | | (9B) | | (9C) | |
| Time | | Mean | CV % | Mean | CV % | Mean | CV % |
| 15 | min | 0.25 | 123 | 0.223 | 90 | 0.067 | 194 |
| 30 | min | 0.436 | 107 | 0.172 | 92 | 0.092 | 195 |
| 1 | h | 0.451 | 109 | 0.173 | 84 | 0.098 | 117 |
| 2 | h | 0.319 | 125 | 0.114 | 68 | 0.061 | 100 |
| 4 | h | 0.158 | 119 | 0.061 | 67 | 0.018 | 198 |
| 6 | h | 0.082 | 118 | 0.012 | 418 | 0.024 | 115 |

A comparison of PK obtained for Formulations 9A, 9B, and 9C is shown in FIG. 10, and also shown in Table 17 below:

TABLE 17
Comparison of the AUClast and Cmax
between different EDTA amount.

| | 50 mg EDTA- | 100 mg EDTA- | 200 mg EDTA- |
|---|---|---|---|
| | Na₂•2H₂O | Na₂•2H₂O | Na₂•2H₂O |
| | (9A) | (9B) | (9C) |
| Cmax (CV %) | 0.451 (109) | 0.223 (90) | 0.098 (195) |
| AUClast (CV %) | 1.443 (112) | 0.556 (70) | 0.277 (135) |

The results above show that the 50 mg of EDTA achieved higher exposure compared to EDTA at 100 mg or 200 mg.

The PK results obtained for se injection are shown in Table 18 below.

TABLE 18
The PK data for studies of sc injection

sc 8 mg injection

| | | |
|---|---|---|
| Time | Mean | CV % |

| | | | |
|---|---|---|---|
| 15 | min | 0.195 | 19 |
| 30 | min | 0.329 | 9 |
| 45 | min | 0.395 | 17 |
| 1 | h | 0.476 | 5 |
| 2 | h | 0.460 | 23 |
| 4 | h | 0.294 | 53 |
| 6 | h | 0.152 | 47 |

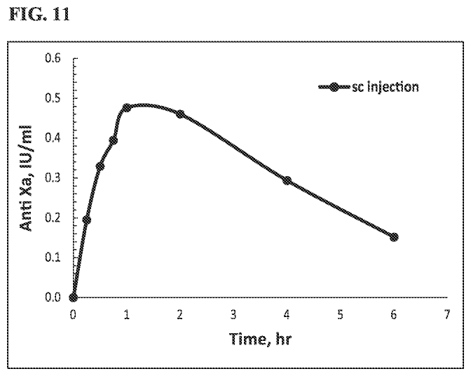

The PK curve for sc injection is shown in FIG. 11, and the Cmax and AUClast for sc injection also shown in Table 19 below:

TABLE 19
The AUClast and Cmax for sc injection.

| | sc injection |
|---|---|
| Cmax (CV %) | 0.476 (5) |
| AUClast (CV %) | 1.957 (28) |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A pharmaceutical composition comprising:
a low molecular weight heparin (LWMH);
an aliphatic acid of Formula I: RCOOH, wherein R represents an aliphatic group, or a pharmaceutically acceptable salt thereof; and
a compound of Formula II: 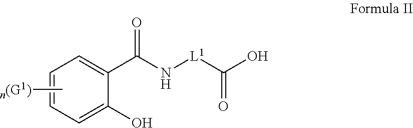
or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ alkyl), halogen (e.g., Cl), C₁₋₄ alkyl, or C₁₋₄ alkoxy (e.g., OCH₃); and
L¹ is a substituted or unsubstituted C₂-C₁₆ alkylene, or substituted or unsubstituted C₂-C₁₆ alkenylene.

2. The pharmaceutical composition of claim 1, formulated for oral administration.

3. The pharmaceutical composition of claim 1, which upon oral administration to a human subject in need thereof, delivers a therapeutically effective amount of the LWMH to the human subject.

4. The pharmaceutical composition of claim 1, wherein in Formula I, R represents an alkyl group having 1-30 carbon atoms, e.g., R is —(CH₂)₁₋₁₈CH₃.

5. (canceled)

6. (canceled)

7. (canceled)

8. (canceled)

9. (canceled)

10. (canceled)

11. (canceled)

12. (canceled)

13. (canceled)

14. (canceled)

15. The pharmaceutical composition of claim 1, wherein the compound of Formula II is

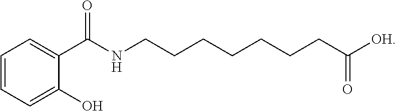 [SMILES: O=C(O)CCCCCCCNC(=O)c1ccccc1O]

16. The pharmaceutical composition of claim 1, wherein the compound of Formula II is

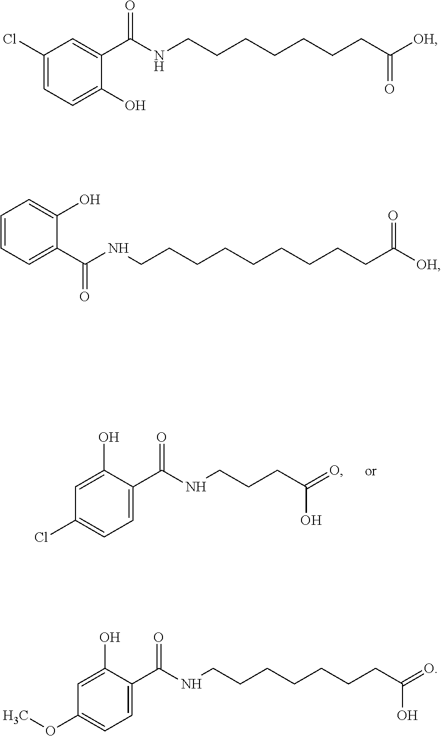 [SMILES: O=C(O)CCCCCCCNC(=O)c1cc(Cl)ccc1O ; O=C(O)CCCCCCCCCNC(=O)c1ccccc1O ; O=C(O)CCCNC(=O)c1ccc(Cl)cc1O ; COc1ccc(C(=O)NCCCCCCCC(=O)O)c(O)c1]

17. (canceled)

18. The pharmaceutical composition of claim 1, comprising a sodium salt of

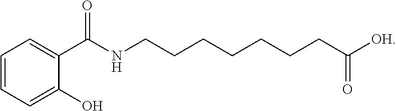 [SMILES: O=C(O)CCCCCCCNC(=O)c1ccccc1O]

19. The pharmaceutical composition of claim 18, wherein the aliphatic acid of Formula I is capric acid.

20. The pharmaceutical composition of claim 1, wherein the weight ratio of (b) the aliphatic acid of Formula I or pharmaceutically acceptable salt thereof to (c) the compound of Formula II or pharmaceutically acceptable salt thereof, (b)/(c), ranges from about 20:1 to about 1:20, such as 5:1 to 1:5, e.g., about 2:1 to about 1:2, such as about 2:1, about 1:1, or about 1:2, or any range in between.

21. (canceled)

22. (canceled)

23. (canceled)

24. The pharmaceutical composition of claim 1, further comprising (d): a chelating agent.

25. (canceled)

26. The pharmaceutical composition of claim 24, wherein the chelating agent is ethylenediaminetetraacetic acid or a salt thereof, such as sodium salt.

27. (canceled)

28. (canceled)

29. (canceled)

30. (canceled)

31. The pharmaceutical composition of claim 1, wherein the LMWH is enoxaparin, bemiparin, nadroparin, reviparin, parnaparin, certoparin, dalteparin, or tinzaparin.

32. (canceled)

33. (canceled)

34. (canceled)

35. (canceled)

36. (canceled)

37. (canceled)

38. A method of treating a disease or disorder for which administering LWMH is beneficial, e.g., deep vein thrombosis) (DVT, Sickle Cell Disease (SCD), pre-thrombotic state/recurrent spontaneous abortion (PTS/RSA), cancer associated thrombosis (CAT), dyslipidemia, etc., in a subject in need thereof, the method comprising orally administering the pharmaceutical composition of claim 1 to deliver a therapeutically effective amount of the LMWH to the subject.

39. A method of preparing the pharmaceutical composition of claim 1, the method comprising (a) mixing the LMWH with the compound of Formula II or pharmaceutically acceptable salt thereof, and the aliphatic acid of Formula I or pharmaceutically acceptable salt thereof to form a mixture; (b) freeze-drying the mixture formed in (a) to form a freeze-dried mixture; and optionally (c) mixing the freeze-dried mixture with a pharmaceutically acceptable excipient.

40. A method of preparing the pharmaceutical composition of claim 24, the method comprising (a) mixing the LMWH with the compound of Formula II or pharmaceutically acceptable salt thereof, and the aliphatic acid of Formula I or pharmaceutically acceptable salt thereof to form a mixture; (b) freeze-drying the mixture formed in (a) to form a freeze-dried mixture; and (c) mixing the freeze-dried mixture with the chelating agent.

41. A method of preparing the pharmaceutical composition of claim 24, as applicable, the method comprising (a) mixing the LMWH with the compound of Formula II or pharmaceutically acceptable salt thereof, the aliphatic acid of Formula I or pharmaceutically acceptable salt thereof, and the chelating agent to form a mixture; (b) freeze-drying the mixture formed in (a) to form a freeze-dried mixture; and optionally (c) mixing the freeze-dried mixture with a pharmaceutically acceptable excipient.

42. A method of preparing a composition comprising a low molecular weight heparin (LMWH), the method comprising:
mixing the LMWH with (i) an aliphatic acid of Formula I: RCOOH, wherein R represents an aliphatic group, or a pharmaceutically acceptable salt thereof; (ii) a compound of Formula II: 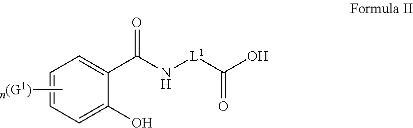
or a pharmaceutically acceptable salt thereof, wherein:
n is an integer selected from 0, 1, 2, 3, or 4;
G¹ at each occurrence is independently OH, NH₂, NH(C₁₋₄ alkyl), N(C₁₋₄ alkyl)(C₁₋₄ C₁₋₄ alkyl), halogen (e.g., Cl), C₁-4 alkyl, or C₁-4 alkoxy (e.g., OCH₃); and
L¹ is a substituted or unsubstituted C₂-C₁₆ alkylene, or substituted or unsubstituted C₂-C₁₆ alkenylene; and optionally (iii) a chelating agent; and
freeze-drying the mixture formed in (a).

43-59. (canceled)
